(19) United States
(12) Patent Application Publication
(10) Pub. No.: US 2021/0038883 A1
(43) Pub. Date: Feb. 11, 2021

(54) AUDITORY IMPLANT

(71) Applicant: THE FRANCIS CRICK INSTITUTE LIMITED, London, Greater London (GB)

(72) Inventors: Romeo-Robert RACZ, London, Greater London (GB); Andreas SCHAEFER, London, Greater London (GB)

(21) Appl. No.: 16/978,054

(22) PCT Filed: Mar. 6, 2019
(86) PCT No.: PCT/GB2019/050620

§ 371 Date: Sep. 3, 2020

(30) Foreign Application Priority Data
Mar. 7, 2018 (GB) 1803667.3

(51) Int. Cl. A61N 1/05; A61N 1/36; A61B 5/145; A61B 5/00

(52) U.S. Cl. CPC A61N 1/0541; A61N 1/36038; A61N 1/36157; A61B 5/686; A61B 5/14539; A61B 5/14532; A61N 1/36153

(57) ABSTRACT

An auditory implant comprises an array of micro-electrodes including at least two stimulation micro-electrodes each comprising a core of conducting material; insulating material surrounding the core; and a layer of metal or metal oxide nano-structures deposited on tips of the micro-electrodes at a first end for interfacing with a target site for auditory stimulation; and an integrated circuit to control a pattern of stimulation current driven through the array of micro-electrodes.

The present technique relates to the field of auditory implants.

Hearing impairment is a sensory loss with major negative impact on the individual's quality of life. Various different treatments are available depending on the extent, type and configuration of the hearing impairment. Potential treatment options include direct electrical stimulation of healthy parts of the auditory pathway, for example as in cochlear implants. Depending on the healthy parts of the auditory pathway, cochlear nerve, auditory brainstem and the auditory cortex may represent feasible stimulation targets.

At least some examples provide an auditory implant comprising: an array of micro-electrodes including at least two stimulation micro-electrodes each comprising: a core of conducting material; insulating material surrounding the core; and a layer of metal or metal oxide nano-structures deposited on tips of the micro-electrodes at a first end for interfacing with a target site for auditory stimulation; and an integrated circuit to control a pattern of stimulation current driven through the at least two stimulation micro-electrodes.

Further aspects, features and advantages of the present technique will be apparent from the following description of examples, which is to be read in conjunction with the accompanying drawings, in which:

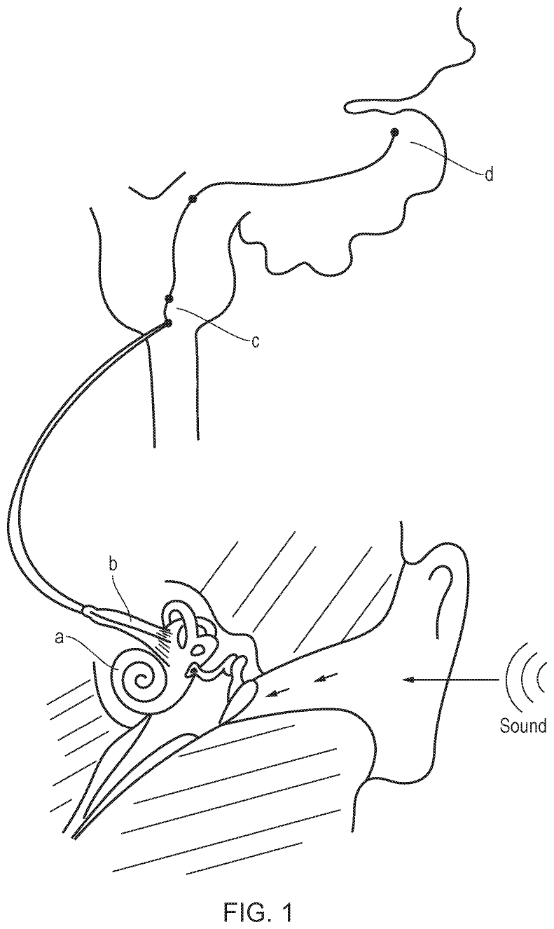

FIG. 1 is a schematic illustration of an auditory path, where potential locations of stimulation target site are shown;

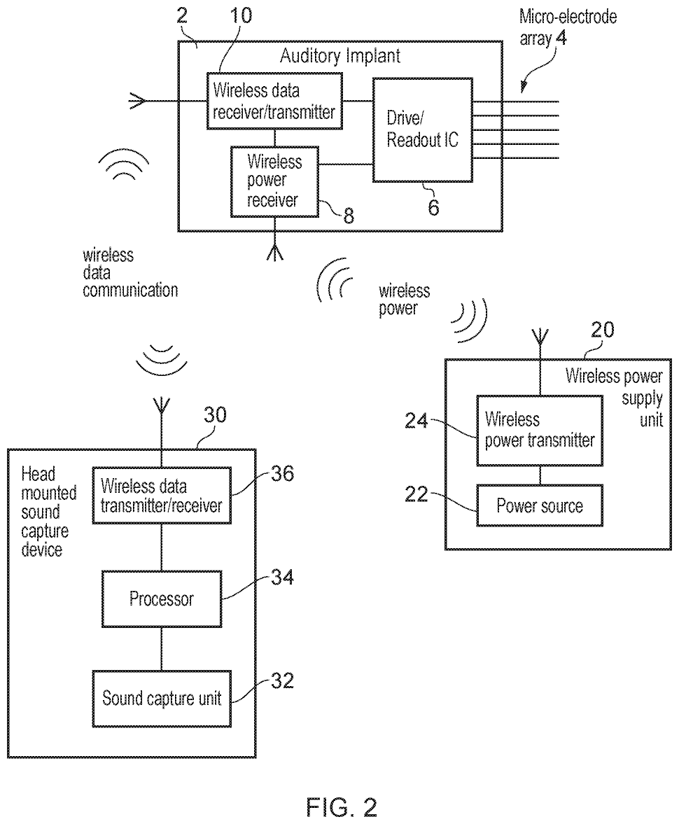

FIG. 2 illustrates a first example of a system comprising an auditory implant, in which the auditory implant receives sound information transmitted wirelessly from a head mounted sound capture device;

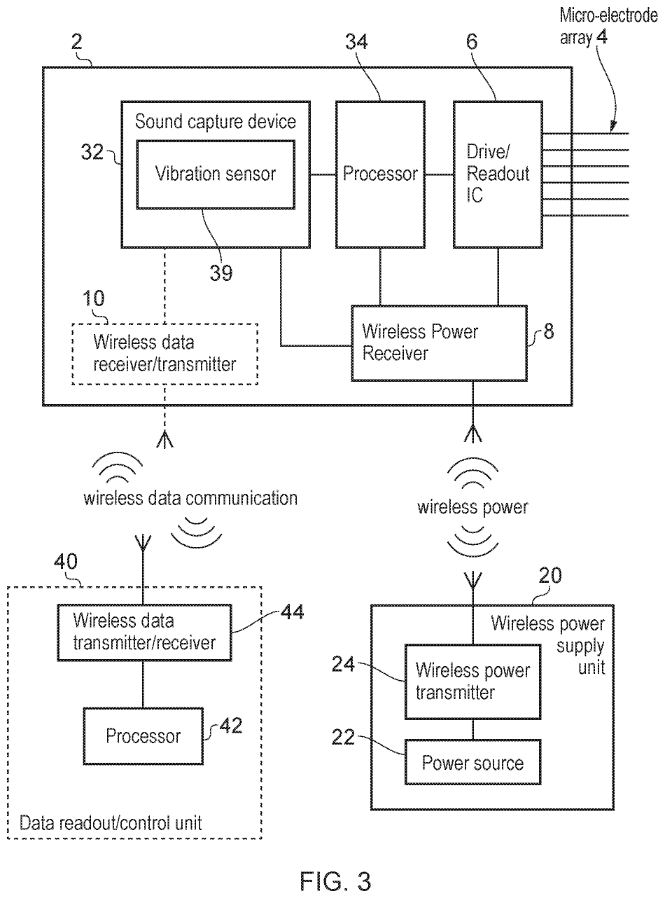

FIG. 3 illustrates a second example of an auditory implant comprising an internal sound capture device;

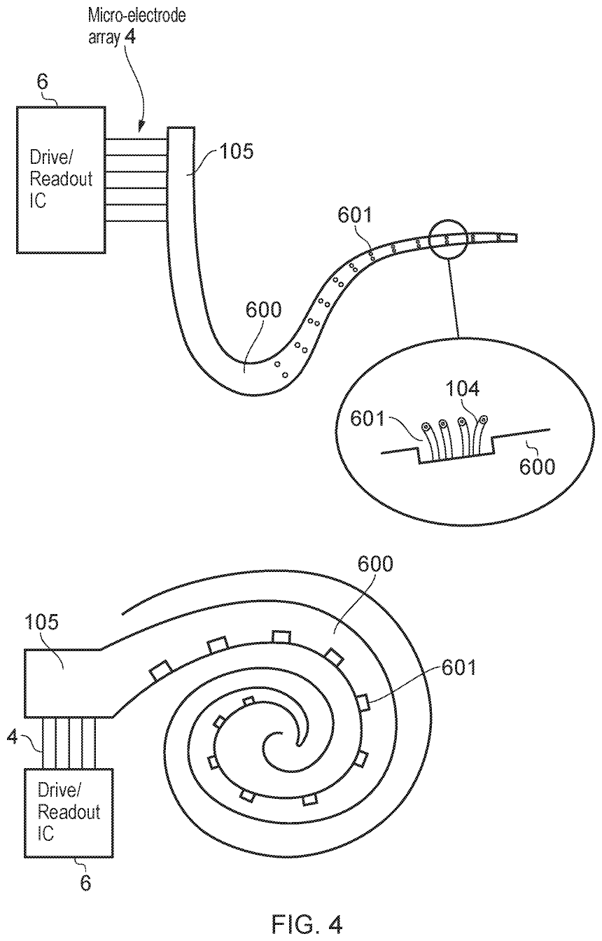

FIG. 4 shows two schematic illustrations of a micro-electrode array used in the implant;

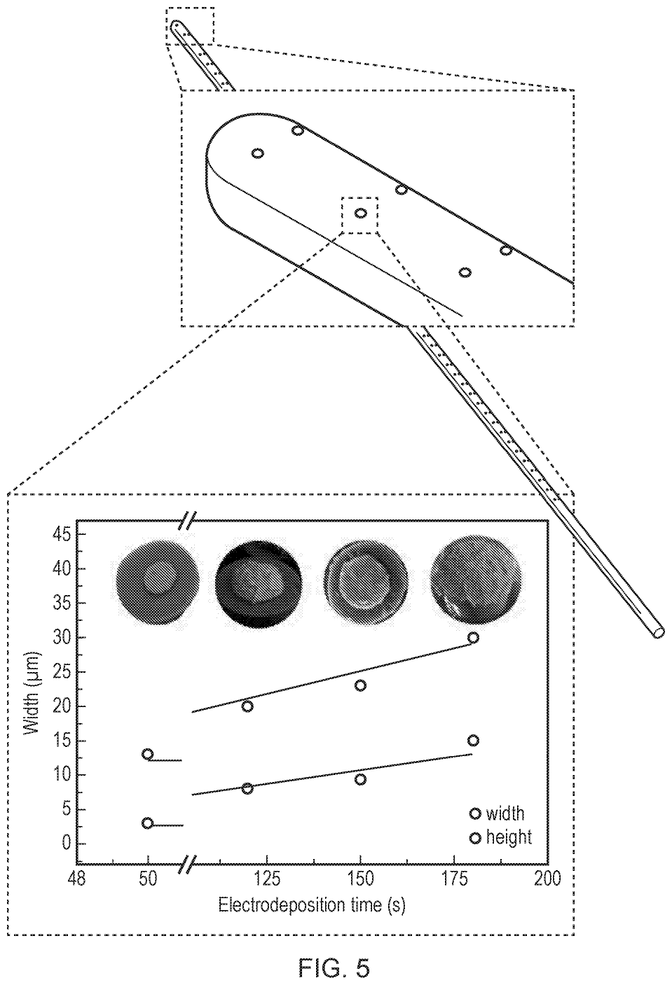

FIG. 5 shows another view of the micro-electrode array used in a cochlear implant, where enlarged examples of the stimulation sites are illustrated;

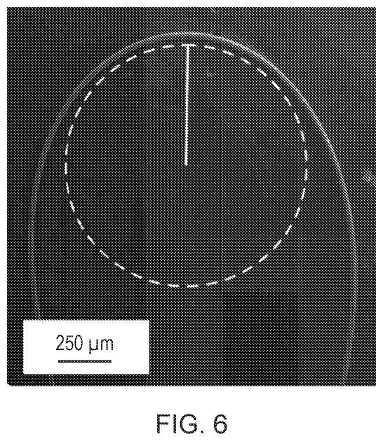

FIG. 6 illustrates a bend radius of one of the micro-electrodes;

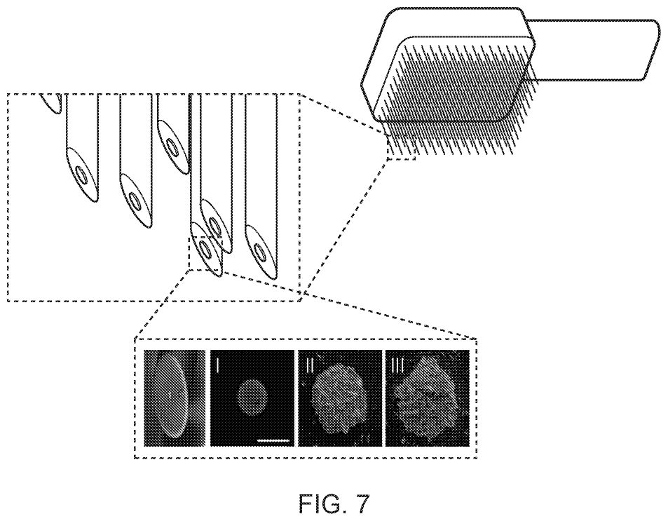

FIG. 7 illustrates another example of a micro-electrode array used in the implant;

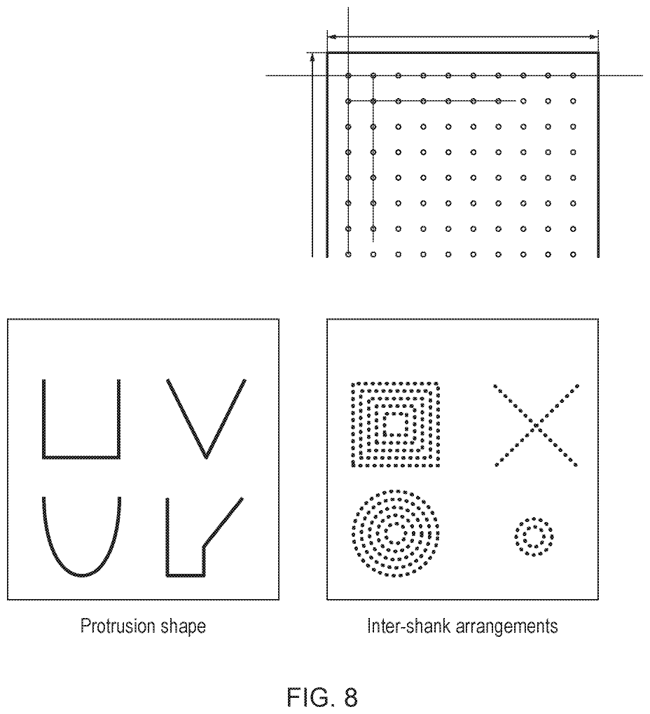

FIG. 8 shows example arrangements of the micro-electrode array in the example illustrated in FIG. 7;

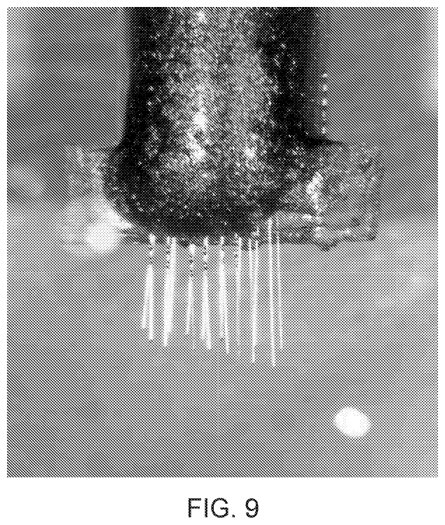

FIG. 9 shows various views of another example of a micro-electrode array used in the implant;

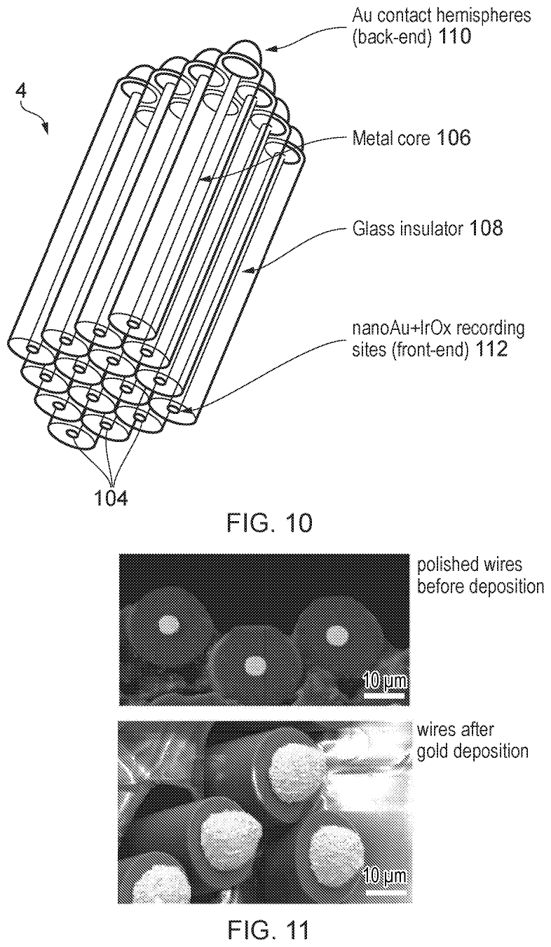

FIG. 10 schematically illustrates an example of an array of micro-electrodes (also referred to as wires) of conducting material surrounded in insulating material with impedance reducing layers made of gold nano-structures deposited on the tips of the wires at the first and second ends, and an iridium oxide functionalization layer deposited on the gold nano-structures at the first end of the wires;

FIG. 11 shows an image of the wires before and after depositing the gold nano-structures;

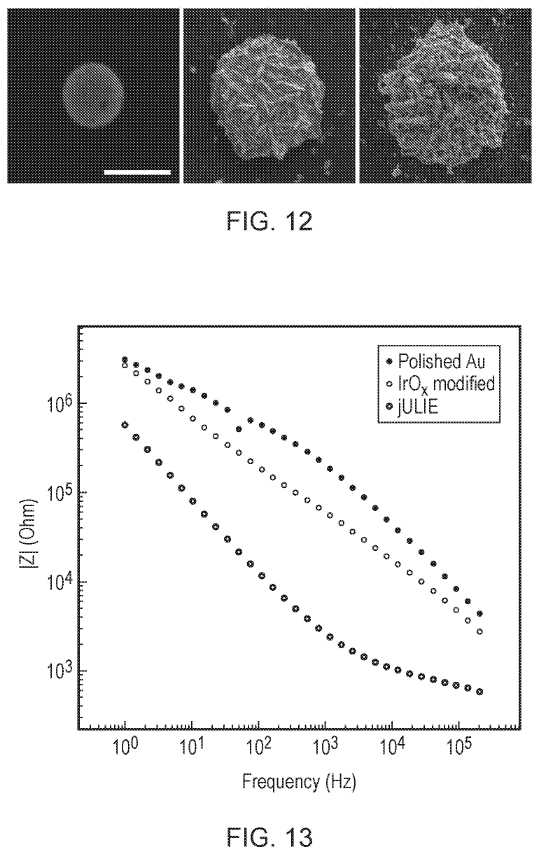

FIG. 12 shows images of the bare metal core wire, the tip of the wire after depositing the gold nano-structures and the tip after depositing the iridium oxide functionalization layer on the gold nano-structures;

FIG. 13 is a graph showing how the impedance at the first end interface of the wires is reduced by including the layer of gold nano-structures;

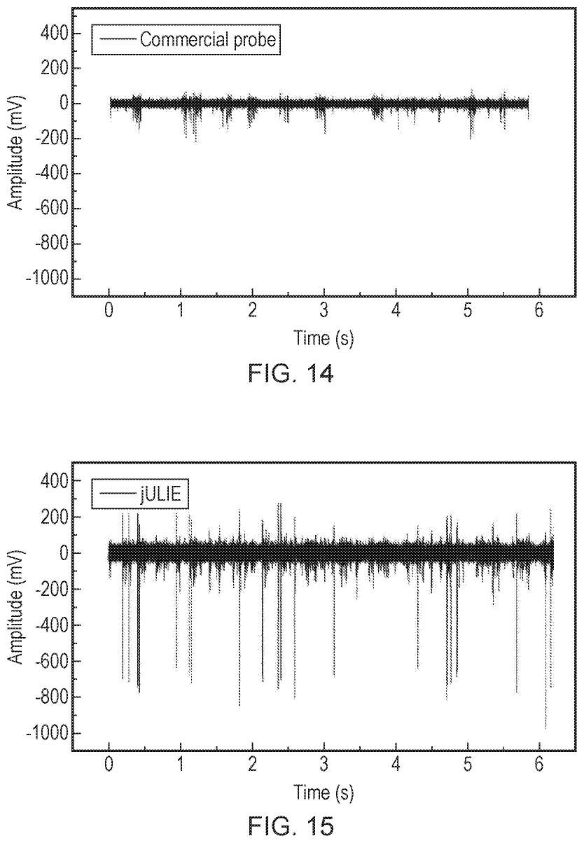

FIGS. 14 and 15 compare signal amplitudes of neuronal recordings measured in a mouse brain using wires of a typical commercial probe and the micro-electrodes of FIG. 1 respectively;

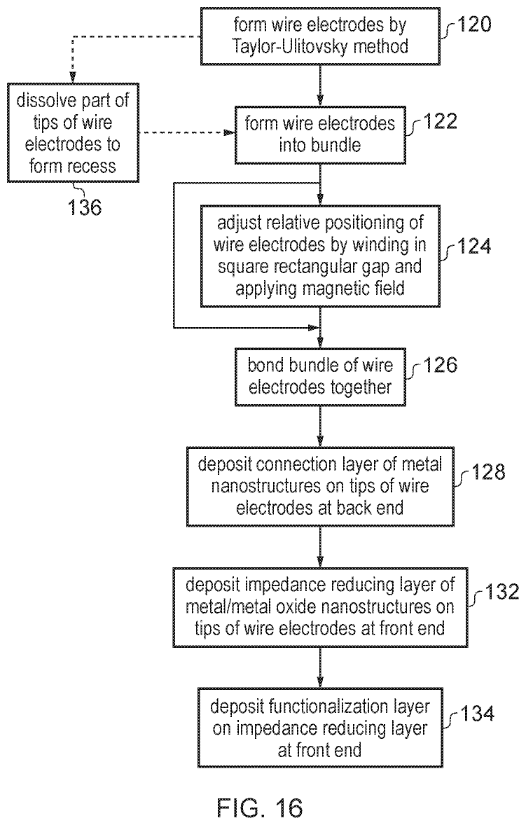

FIG. 16 is a flow diagram illustrating a method of manufacturing the array of micro-electrodes;

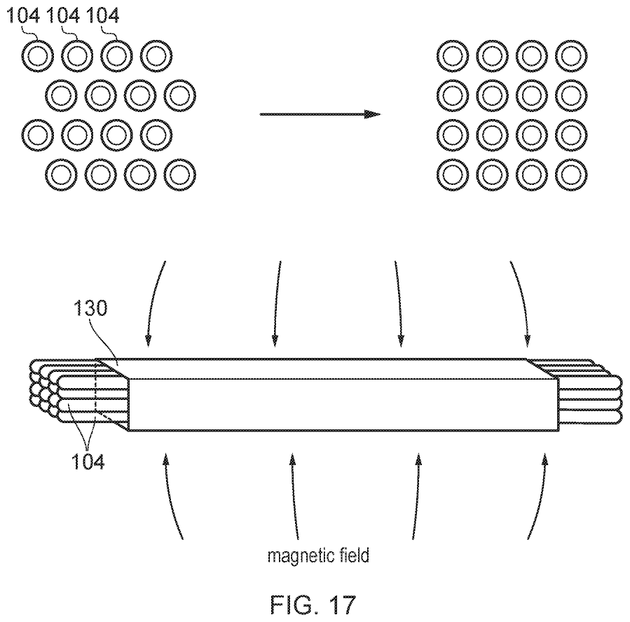

FIG. 17 schematically illustrates an example of adjusting the relative positioning of the micro-electrodes in the array using a magnetic field;

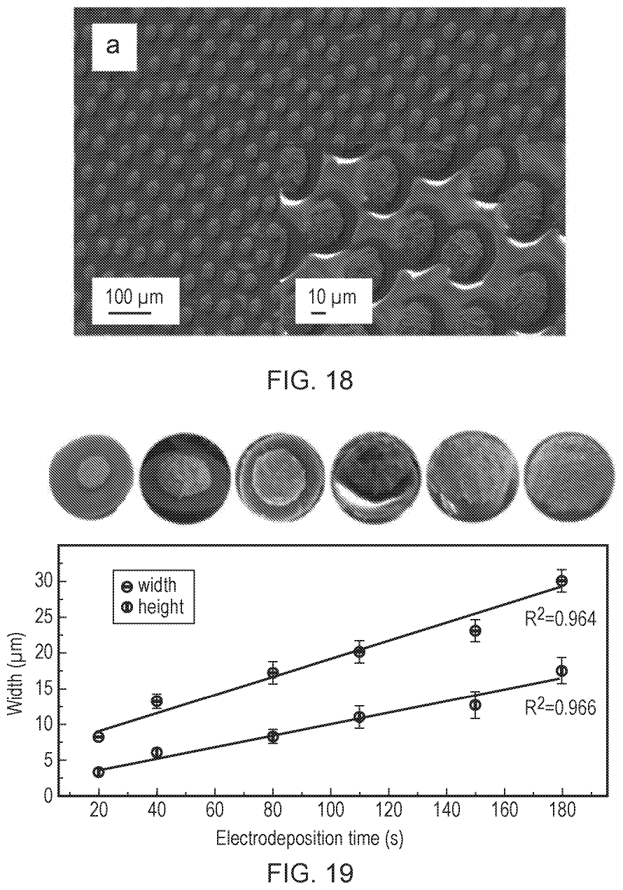

FIG. 18 is an image showing an example where the insulating sheathes of the electrodes are melted together to bond the wires together in the bundle;

FIG. 19 is a graph showing variation of the size of gold nano-structure bumps with electrodeposition time;

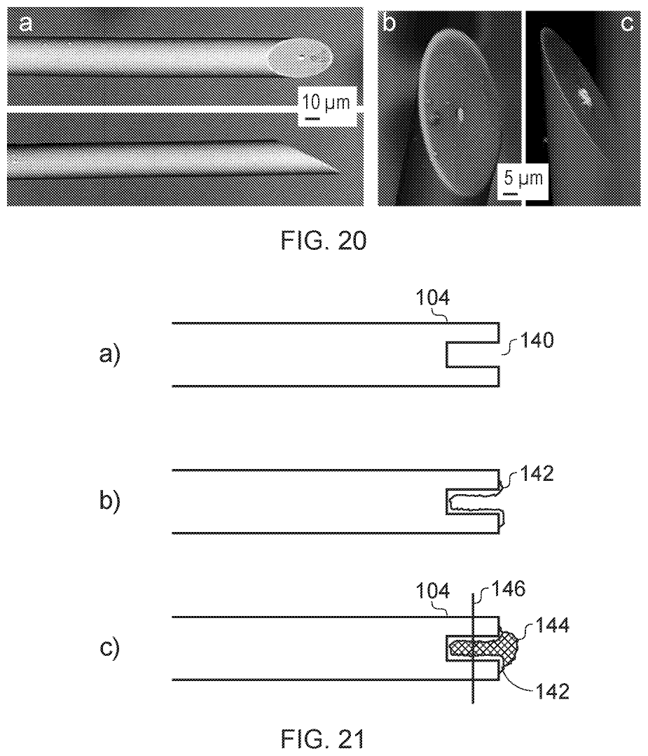

FIG. 20 shows an example of sharpened tips of the electrodes;

FIG. 21 shows an example where a recess is formed in the tips of the electrodes and the impedance reducing layer and functionalization layer are deposited on the inside of the recess;

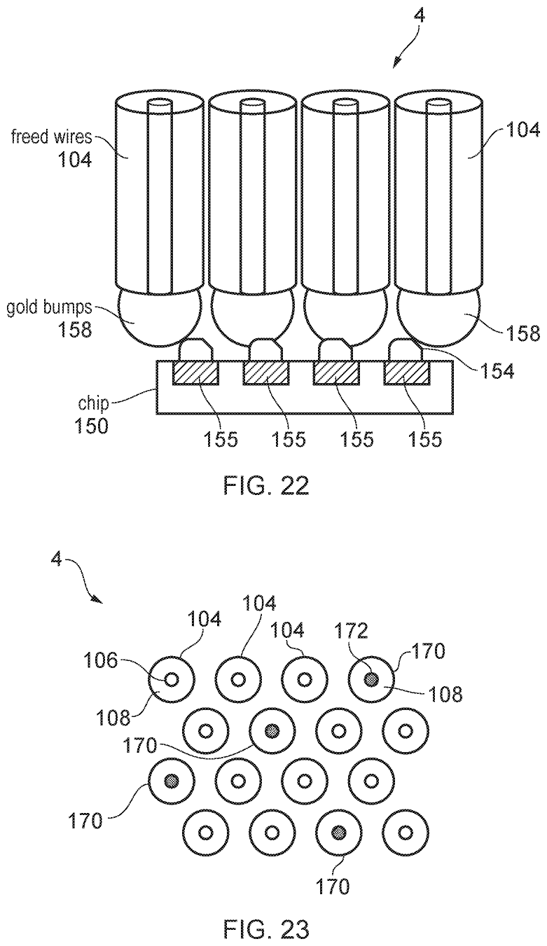

FIG. 22 shows an example of an apparatus in which an integrated circuit is used to drive stimulation currents through at least a subset of the electrodes, and optionally read out and amplify the signals received from at least a subset of the electrodes;

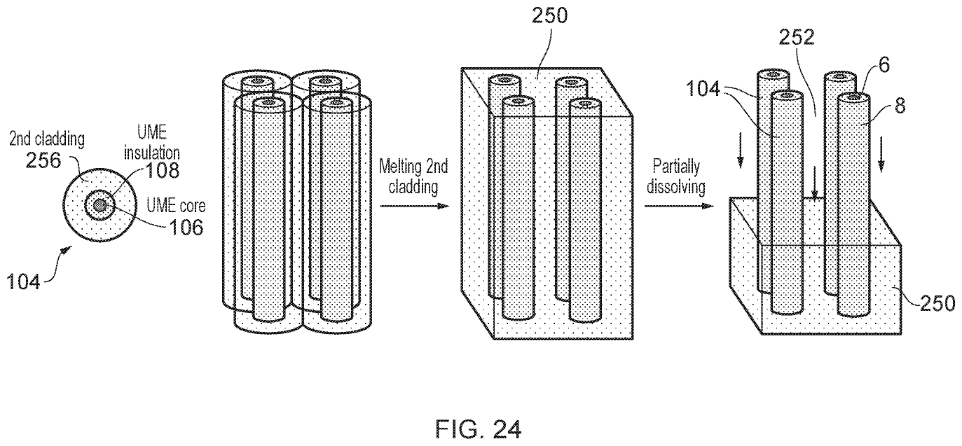

FIG. 23 shows an example of providing at least one hollow-core channel in parallel to the micro-electrodes;

FIG. 24 shows an example of manufacturing electrodes partially embedded in cladding material along part of the length of the electrodes;

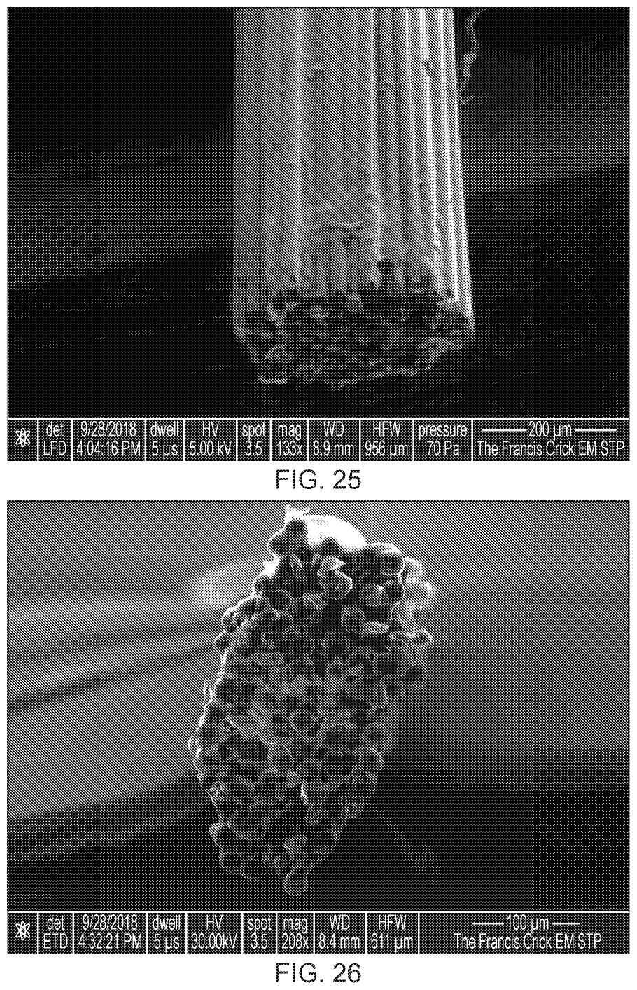

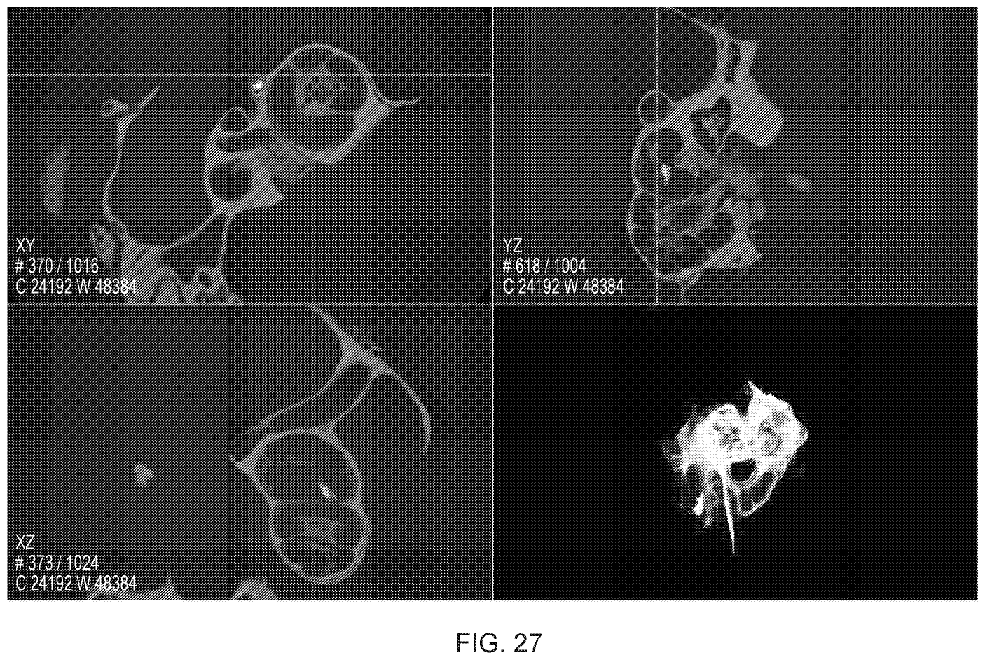

FIGS. 25 and 26 show SEM micrographs showing side and front views of a cochlear implant with approximately 100 channels;

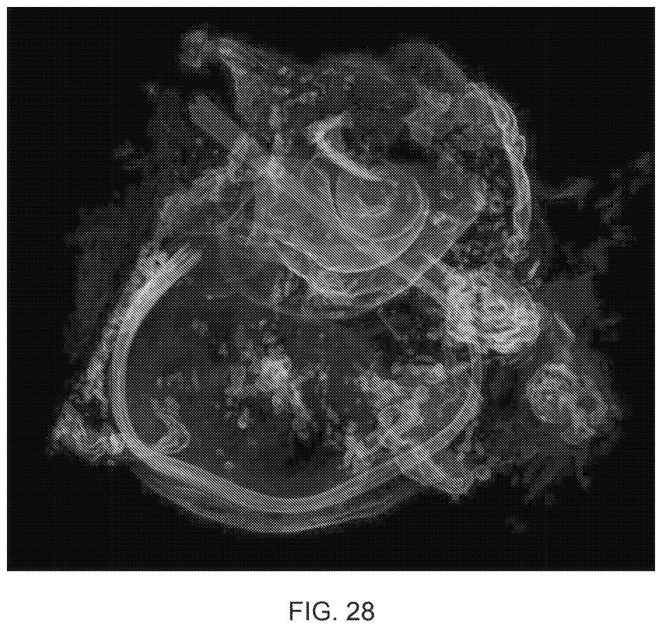

FIG. 27 shows micro-CT micrographs showing the ˜100-channel implant inserted in the base of a gerbil cochlea; and

FIG. 28 shows a micro-CT micrograph showing an implant with approximately 1000 channels inserted in a sheep tympanic bulla.

An auditory implant comprises an array of micro-electrodes which include at least two stimulation micro-electrodes. Each of the stimulation micro-electrodes comprises a core of conducting material, insulating material surrounding the core, and a layer of metal or metal oxide nano-structures deposited on tips of the micro-electrodes at a first end for interfacing with a target site for auditory stimulation. The auditory implant also includes an integrated circuit to control a pattern of stimulation current driven through the array of micro-electrodes.

There is an acute need for miniaturisation of electronic devices which come in contact with nervous tissue as biocompatibility is governed by the lack or presence of biological reactions which when present will damage the device and cause medical complications. A problem with the miniaturization of existing devices for auditory stimulation has been that the electrodes when miniaturized will typically have a relatively high impedance at the interface between the electrode and the tissue being stimulated, and so this has often necessitated use of relatively high currents and additional circuitry for amplifying stimulation currents. A disadvantage of using higher currents is that this may cause more tissue damage, and also the additional amplification required may increase the power consumption of the device. Another disadvantage of using higher currents is decreased accuracy. Higher currents are more likely to stimulate a larger area of target site, which means that those that are not relevant may be stimulated. Thus, target site can be more accurately stimulated using lower currents.

In contrast, the auditory implant discussed in the examples below has a layer of metal or metal oxide nano-structures deposited on tips of the micro-electrodes at the first end for interfacing with the stimulation target site. This helps to reduce impedance at the first end of the micro-electrodes, hence reducing the currents required thus helping to reduce the likelihood of tissue damage and reduce power consumption of the implant. Additionally, the use of lower currents enables more accurately stimulating the intended stimulation target site. Hence, this implant can act as a more effective tool for supporting research into the way in which auditory stimulation by applying stimulation currents from electrodes is perceived in terms of a sound by the brain, enabling better research into the detailed understanding of how the auditory system functions. Also, such an auditory implant can also be more effective for restoring hearing in individuals with sensory loss. If the implant is used as a research tool for example, then this could be used to drive the electrodes with certain test patterns of stimulation current and then the effect of each test pattern can be studied, either by asking a human individual to describe the auditory sensations experienced when certain patterns of stimulation currents are applied, or by using a second implant to sense changes in a human or animal's brain caused by applying particular patterns of stimulation current.

The micro-electrodes may have a diameter at the micron scale. More particularly the micro-electrodes may have a diameter less than or equal to 30 μm; or less than or equal to 25 μm, or less than or equal to 20 μm, or less than or equal to 15 μm, or less than or equal to 10 μm. Given the micron scale of the electrodes, this means that an implant may have much higher channel count compared to current techniques which use wider electrodes having larger diameters. This means that a higher resolution reading or stimulation can be achieved using micro-electrodes. Hence, by using extremely narrow micro-electrodes formed using a conducting core and an insulating surrounding, this can provide a much improved platform for enabling better research into the auditory sensation in humans or animals and eventually better implants for hearing restoration. Another advantage of using narrower micro-electrodes is that, because the current at each location stimulated by an individual electrode is much lower, and heating of tissue is proportional to the square of the current, the amount of tissue damage can be much less than if fewer electrodes with larger widths and larger currents were used.

Yet another advantage of using narrower micro-electrodes is that, due to the size of the micro-electrode, it is possible to insert single or multiple micro-electrodes to a tissue without causing significant damage to the tissue. For example, with known electrodes, it is often the case that the larger size breaks and damages the tissue surrounding the electrode. On the contrary, when a single or multiple micro-electrodes are inserted, they are far more likely to navigate through the tissue in particular pushing past capillaries rather than needing to break through one and thus damaging the tissue in the area surrounding the electrode. Thus, a higher portion of the tissue remains healthy with micro-electrodes. This also means that a lower current is required to achieve stimulation, because healthy cells are closer by. This in turn enables surrounding tissues being maintained in a healthy form. In some examples the number of stimulation micro-electrodes provided may be greater than 100; or greater than 1000; or greater than 10000; or greater than 100000.

The micro-electrodes may be provided as a single array or multiple arrays.

As well as the layer of metal or metal oxide nano-structures on the tips of the micro-electrodes at the first end, which may help to reduce the interface impedance, the at least two stimulation micro-electrodes may also have a connection layer of metal nano-structures deposited on tips of the micro-electrode at a second end for interfacing with the integrated circuit. This may help reduce resistance at the interface between the electrodes and the integrated circuit again reducing losses and hence reducing the amount of amplification required which saves power. The compressible nature of the layer of nano-structures at the second end can also be helpful for improving bonding with the integrated circuit, as the nano-structures when pushed against bumps on the integrated circuit may squash to provide a tight coupling and can squeeze into gaps more easily than solid conducting material.

The nano-structures at the first end may be made from a range of materials but in one example may comprise nano-structures made of a noble metal. For example the noble metal may be gold. Similarly the nano-structures at the second end could also be made of gold or other noble metals.

In some cases, the stimulation micro-electrodes could also comprise a functionalisation layer deposited on the layer of metal or metal oxide nano-structures at the first end. The functionalisation layer may be made from a range of materials such as inorganic materials such as metal oxides, e.g. iridium oxide or copper oxide, or other substances such as DNA, graphene, carbon nanotubes, antibodies, aptamers, enzymes etc. The functionalisation layer may support improved delivery of stimulation current through the micro-electrodes (e.g. a layer of iridium oxide may boost charge capacity to improve current injection), and/or may support other diagnostic or sensing functions, e.g. targeting particular biomarkers.

Various insulators could be used to surround the conducting core of the micro-electrodes, but in one example the insulating material surrounding the core in the stimulation micro-electrodes may be glass. Glass-unsheathed metal micro-electrodes can represent an ideal platform for high channel count interfacing with elements of the auditory pathway as this provides a biocompatible and aseptic material which permits formation of mechanically flexible electrodes, which can be advantageous to reduce tissue damage when the electrodes are inserted. In one example, the micro-electrodes may comprise “microwires” or Taylor-Ulitovsky wires, formed using the Taylor-Ulitovsky method. The Taylor-Ulitovsky method is a technique for forming conducting wires surrounded by glass, with extremely narrow widths. It has been found that Taylor-Ulitovsky wires have extremely low impedance when treated with the layer of nano-structures on the first end tips providing an extremely versatile, biocompatible and precision instrument for exploring the phenomenon of vision by supporting experimental research into auditory stimulation and implementing auditory implants for hearing restoration. Also, Taylor-Ulitovsky wires have extremely smooth sides, reducing the likelihood of surface roughness causing tissue damage as the electrodes are inserted or removed.

The micro-electrodes may have a bending radius of at least 500 μm. Such flexibility also enables the array of micro-electrodes (or a bundle of electrodes) to bend and twist into required shapes where required.

In one example, at least two protruding micro-electrodes at the first end may have different lengths. The micro-electrodes may be protruding away from the main array body, in which the lengths of the protruding portions of the micro-electrodes may differ. The micro-electrodes differing in lengths may both be stimulating micro-electrodes, in which case the micro-electrodes of different lengths may be positioned such that the target site may be more efficiently stimulated when implanted. For example, the target site may have different depths (e.g. in the brain, or in a nerve bundle), where different lengths could be used to reflect the different portion of the brain or nerve bundle that need to be stimulated. Alternatively, or in addition to this, some micro-electrodes may be stimulating micro-electrodes and some may be sensing micro-electrodes.

In one example, the tips of the micro-electrodes at the first end may have a polished angled tip surface with a sharpened point, and the at least two stimulation micro-electrodes may have the nano-structures formed on the angled tip surface. The angled tip helps facilitate better insertion into the sample, reducing the amount of tissue damage. The tips of the micro-electrodes at the first end may have other shapes, such as flat tip surface which angled in relation to the length of the micro-electrode, for example perpendicular to the length of the micro-electrode, a tip surface which comprises a plurality of flat surfaces so as to have a polygon-like shape), or a curved surface. Differently shaped tips may be provided depending on the function of the micro-electrode, and the intended location of the micro-electrode at the target stimulation site.

The integrated circuit for driving the stimulation current through the micro-electrodes can be implemented in different ways. For example, integrated circuit of commercially available cochlear implant may be used, or bespoke integrated circuits could be designed. Even in examples in which the intended stimulation target site of the micro-electrode is not in the cochlear, the integrated circuit of commercially available cochlear implant may be used.

The bonding between individual micro-electrodes and corresponding pixelated contact portions of the integrated circuit could be done with individual metallic/conducting bonds being formed between the micro-electrodes and the individual unit of the pixelated contact portion. However, when the channel count is particularly high, and given the small size of the micro-electrodes, in some cases there may not be a specific bonded connection formed in the manufacturing process. Instead, in one example the second ends of the micro-electrodes can simply be pushed up against the pixelated contact portion of the integrated circuit and the connection between particular units and individual electrodes can simply be formed by the contact between the two. The second end metal nano-structures discussed above can be particularly helpful in allowing such connections to be formed, since the nano-structure of the second end bumps can be squashed on impact with the corresponding pixel unit to form a better connection. This is discussed in more detail below.

In other examples, the integrated circuit could also include some units which measure currents detected through some of the electrodes. In this case, in addition to units for driving currents there could also be a separate unit with amplification for reading out current. In other examples, the integrated circuit could also further include some units which are configured to activate other functions of the implant, for example to deliver light, including light in the visible and infrared spectrum, or to deliver microfluidics through hollow-core channels that may be provided in addition to the micro-electrodes in the array.

In one example, the integrated circuit may perform simultaneous driving of currents through stimulation micro-electrodes of the array arranged in a two dimensional pattern. That is, rather than sequential raster-scanning across the two-dimensional array in a series of one dimensional lines, the integrated circuit may be designed to simultaneously apply current through different micro-electrodes extending over two dimensions simultaneously. A simultaneous driving mode can be useful for an auditory implant, because raster-scanning sequentially may cause synchronicity problems in the brain.

In some examples, an arbitrarily selected pattern of stimulation current may be selected to be driven through the array of micro-electrodes. For example, for running a particular test or research experiment, then certain stimulation patterns could be devised and communicated to the integrated circuit (or generated within the integrated circuit itself), and then such currents could be applied by the stimulation electrodes, and feedback can be gathered on the effects of such stimulation, either by asking the individual in which the implant is implanted for a description of the auditory sensations experienced, or by using other probes to measure brain function in response to the stimulation applied. This could then allow improved knowledge to be gained regarding the particular auditory sensations elicited in response to certain stimuli, which could help for the subsequent development of auditory implants for hearing restoration.

However, in other examples the integrated circuit may receive sound information indicative of a captured sound and control the pattern of stimulation current based on the received sound information. This could be useful for enabling the user's surroundings to be perceived auditory by a deaf or partially hearing impaired user using the auditory implant. In some examples, the sound capture unit which captures the sound may not be part of the implant itself. Instead, the implant may comprise a wireless communication unit which receives the sound information from an external sound capture device. This can reduce the complexity of the implant. For example, a head mounted sound capture unit could be provided, which could be worn on the head of the user to capture a sound. For example, the sound capture unit may be worn around the ear of the user to approximately represent the sound received around the location of the user's ears. The captured sound can be used to derive the information to transmit to the integrated circuit within the implant. The integrated circuit in the implant can then drive a corresponding pattern of stimulation current based on the received sound information.

In other examples, the implant itself may comprise a sound capture unit to capture the sound, and processing circuitry to process the captured sound to generate the sound information based on the captured sound. For example, the sound capture unit may be connected to the middle ear portion. This processing circuitry could in some cases be implemented on the same integrated circuit that drives the current through the electrodes, or alternatively could be on a separate integrated circuit. With this approach, the sound capture system may be connected to the middle ear portion, for example, and be positioned internally within the skull of the user. This could be useful to reduce the inconvenience caused to the user by having to wear the head mounted sound capture unit. Using an internal sound capture unit (e.g. middle ear vibration sensor) also means that less distortions are introduced to the sound information captured, for example as no microphones need to be used where such microphones may distort sound or amplify wind.

In some examples, the auditory implant may include processing circuitry to detect features in the captured sound indicated by the sound information and to determine a pattern of stimulation current to be applied to the at least two stimulation micro-electrodes based on the detected features. For example the sound detected may be in various different frequencies and considering which frequencies normally fall within human speech, for example, other frequencies may be filtered to be reduced. The features could comprise frequency, amplitude, duration and direction. The duration refers to how long a certain sound is considered to continue for. The direction could be determined, for example, using a plurality of sound capture devices in addition to the sound information determined from the sound capture device. For example, depending on which sound capture device receives a sound which is predicted to be from a single source more loudly or quickly, the direction of the sound may be determined.

Depending on the sound detected, the stimulation could be modulated. For example, the processing circuitry could modulate at least one of the spatial position, frequency and intensity of the stimulation current applied to particular stimulation micro-electrodes based on the detected features

Again, providing processing circuitry of this form to derive patterns of stimulation from captured sounds can be useful in providing a platform for research enabling researchers to explore how to map features of captured sounds into stimulation currents to be applied and requesting feedback from the patient on what auditory sensations have been experienced. Also, eventually auditory implants can be provided to use the best identified approaches for mapping sounds into corresponding stimulation current patterns in order to provide an implant permitting restoration of auditory function. The processing circuitry which detects the features in the captured sound could be located either within the same implant that is to go into the patient, or as externally provided processing circuitry (for example within the same unit as the sound capture unit in embodiments where the sound capture takes place outside the body).

In some examples, the array of electrodes may comprise only the stimulation micro-electrodes so that all of the electrodes are for applying stimulation currents.

However, in other embodiments the array may also comprise at least one sensing micro-electrode and the integrated circuit may control supply of current to, or read out of voltage or current from, the at least one sensing micro-electrode for sensing a predetermined substance or condition. As the implant may provide electrodes which extend into portions of the auditory system such as the cochlear nerve bundle, brainstem or auditory cortex within the brain for example, this also provide an opportunity for diagnostic information to be derived from measurements taken by some of the electrodes. These measurements could be performed for the purpose of identifying responses to the stimulation current supplied by some of the electrodes, or could be for performing sensing independent of stimulation. For example, one cause of loss of hearing may be diabetes and so it may be useful to use the implant for sensing the progression of diabetes or any biomarkers associated with this. For example, it may be useful to sense at least one of glucose or pH (protons, which may be a bio marker for, for example, diabetes or other conditions which are a of change in acid-base physiology) and so the at least one sensing micro-electrode could comprise at least one glucose or pH sensing micro-electrodes. For example the sensing micro-electrodes provided for glucose or pH sensing may include, deposited on the layer of metal or metal oxide nano-structures at the first end of the electrodes, an additional functionalisation layer which may comprise at least one of iridium oxide, copper oxide or graphene, which can all assist with glucose or pH sensing. Another example can be to provide sensing micro-electrodes for sensing progression of Alzheimer's disease, based on detection of an appropriate biomarker in the stimulation location of the auditory path. Also, it would be possible to detect glial fibrillary acidic protein (GFAP) in traumatic brain injuries and brain tumours (glioblastoma) by decorating the nano-structures with anti-GFAP antibody. Other types of functionalisation layer could include other inorganic compounds, enzymes, carbon nanotubes, aptamers, antibodies, or nucleic acid such as DNA or RNA, cDNA, mRNA.

Hence, if the array also includes sensing micro-electrodes then the integrated circuit may also comprise at least one read out circuit for detecting and amplifying a current measured through the at least one sensing micro-electrode. Also wireless communication circuitry may be provided to transmit detection results obtained from the at least one sensing micro-electrode to an external device. These readouts can then be useful in diagnosing conditions in the patient. In some examples at least one micro-electrode of the array may function as both a stimulation micro-electrode and a sensing micro-electrode. For example sometimes current could be applied through an electrode and at other times current may be read out through the electrode. In this case, the corresponding element of the integrated circuit may require both drive capability and readout capability. Other examples may have a separation between the electrodes used for stimulation and the electrodes used for sensing. For a research tool, it can be useful to provide for both stimulation and sensing capability. For a practical auditory implant, it may be preferred to increase the channel count available for stimulation by not providing any sensing micro-electrodes or by making each stimulation micro-electrode dual function (stimulation and sensing).

The implant may comprise a mechanically flexible tubular body carrying the micro-electrodes, where the plurality of the micro-electrodes protrude out of the tubular body at individual stimulation sites located along the tubular body. This example is particularly useful when the implant is intended for the cochlea, as the flexible tubular body can be placed within the cochlea which is spiral shaped. For example, the second ends of the micro-electrodes may be connected to the integrated circuit, and the bodies of the micro-electrodes may be bundled together through the length of the flexible tubular body, where the tips of the micro-electrode at the first end protrude out of the flexible tubular body at stimulation sites. The stimulation site may be aligned with the target stimulation site when the implant is implanted. At each stimulation site, a plurality of tips of the micro-electrodes may be protruding out of the tubular body. The flexible body may have shapes other than tubular, such that the cross-sectional shape is not circular but rectangular, for example. Other cross-sectional shapes are also possible. For example, each stimulation site may correspond to different frequencies, as in a cochlea.

The array of microelectrodes may comprise additional hollow-core channels arranged in parallel with the micro-electrodes. The hollow-core channels may comprise a hollow core surrounded by the insulating material. Such hollow-core channels may be configured to be used as at least one of, or both of micro-fluidic channels and light delivery channels.

The implant may have a removable stylet for aiding insertion of the auditory implant. For example, the stylet could assist the flexible tubular shaped body of the implant be placed around the cochlea.

The protruding tips of the micro-electrodes at the first end at each individual stimulation site may be configured to be spread out. In other words the tips may be fanned out, so as to increase the stimulation area where the tips of the micro-electrodes are present.

The micro-electrodes may be held within a block which extends along part of the length of each micro-electrode. The positions of the second ends of the micro-electrodes can be better controlled in this manner. For example, the body of the micro-electrode may then be placed within the flexible tubular body mentioned above, or in other examples the micro-electrodes may be provided as an array held within a block with first end portions protruding from the block, without any flexible tubular body. These examples are particularly useful when the implant is intended for stimulating nerves in the auditory nerve path that is not located in the cochlea.

The array of micro-electrodes may extend out of the block, where the tips of the micro-electrodes at the front end may form an overall profile which is predetermined based on the shape and location of the stimulation target site. In other words, the overall profile shape may be configured to reflect the anatomical shape of the portion of the auditory nerve pathway that is the target stimulation site. For example, different depths of the brain may need to be stimulated, and to reflect this, the lengths of the micro-electrodes may vary depending on the corresponding portions of the brain that each micro-electrode is intended to stimulate. Such shapes of intended stimulation site may be determined from CT scans, for example. Even if different layers of the brain is not to be stimulated, the lengths of the microelectrode may be varied to better fit the brain curvature and shape of the relevant intended stimulation target site.

The array of micro-electrodes may be provided in a two-dimensional pattern, in which the two-dimensional pattern is predetermined based on the shape and location of the stimulation target site. Again, the two-dimensional pattern may be varied to better fit the brain curvature and shape of the relevant intended stimulation target site. For example, the pattern may include at least one of: a rectangular grid, a circular grid and a cross. Thus, the pattern may be provided such that the tips of the micro-electrodes at the first end can be defined using polar co-ordinates such that the locations of the tips of the micro-electrodes form rings of dotted circles reducing in diameter, or the pattern may be provided such that the tips of the micro-electrodes are provided at the cross-sections of the rectangular grid.

The implant may have sensing micro-electrodes which comprise further reaching tips than other micro-electrodes. For example, the sensing micro-electrodes may be longer than other micro-electrodes. The sensing micro-electrodes having further reaching tips may reach the stimulation site before the other micro-electrodes. The sensing micro-electrodes may have different modalities. The sensing micro-electrodes may output the changes in the implant location such that the stimulation micro-electrodes may be better guided in their positioning.

The implant may have a wireless power receiver for receiving power supplied by wireless communication from an external power source. This avoids the need to provide a complete power source within the implant itself, which would otherwise increase the size of the implant and make it harder to restore power once any battery within the implant has run out without costly operations. In some cases, all of the power required by the implant may be received from external sources by the wireless communication. For example any known radio frequency power technique currently used for medical implants can be used. Alternatively, in some cases the auditory implant could have a battery or other form of charge storage, but by providing means for supplying power to charge the battery from a remote source through wireless communication this means that reduces the chance an implant implanted into a patient needs to be replaced due to batteries running out.

High channel-count auditory prosthetic devices described below combine penetrating metal ultramicroelectrodes (UMEs) arrays with very large scale integrated (VLSI) circuitry to record activity and stimulation. By combining micron-sized metal microwire arrays by bonding with pixelated integrated circuits, a versatile, biocompatible and precision instrument is developed. Protruding microwire arrays can be up to several centimeters long, are mechanically flexible and can be readily inserted into nerves. Furthermore, multiple sensing modalities are provided for the implant device, and furthermore it is scalable.

Using the implant device described below, it is possible to collect sound information from an external device or an internal device and transport it as electrical/biochemical information along the auditory pathway to form the auditory sensations.

Glass-ensheathed metal microelectrodes represent an ideal platform for high channel count and biocompatible interfacing with the numerous elements of the auditory pathway. Sharpened microwires can access cochlear nerves, the brain stem or the auditory cortex causing un-detectable damage to the extracellular matrix or the blood brain barrier.

Here, a prosthetic device is developed from the combination of sharpened metal microelectrodes as interface between electronics and tissue with arrays of amplifiers and/or potentiostats. The latter are drivers for information retrieval (recording) and charge injection (stimulation) into the nerve bundles. By stimulation of a subset of a relevant portion of the auditory nerve path, auditory sensations could be evoked in the form of frequency and volume which form the basis of useful hearing.

Given the micron scale dimensions of the microwires and increasingly high channel count of VLSI electronics, a sensory function restoration apparatus can contain several tens of thousands of contacts for high granularity stimulation without causing damage to the tissue and blood-brain-barrier (BBB). Microwires satisfy the electronic and mechanical requirements for stimulation and recording from neural tissue with single and multi-unit resolution. The prosthesis device may stimulate in a simultaneous manner, as opposed to sequential raster scan that might cause synchronicity problems downstream in the auditory cortex.

Also, given their chemically inert nature and availability of electronic components in the VLSIs, an array of electrochemical sensing elements can be integrated within the same apparatus, for e.g. glucose or pH detection for diagnostic purposes thus developing a versatile, scalable and highly precise prosthetic.

Given the aseptic requirements and volumetric limitations within the skull, two versions have been considered: a wireless enabled design where implanted UMEs receive patterned stimulation information on radio frequencies from a microphone placed on a head-mount and a fully integrated bionic ear with sound acquisition and processing contained within the skull, by accessing sound via vibration through skull or the mechanical portions of the middle-ear.

FIG. 1 is a schematic illustration of an auditory path, where potential locations of stimulation target site are shown.

A very brief and simplified description of a normally functioning ear is as follows. Sound waves are received by the outer ear which then travels along the auditory canal and causes the tympanic membrane, the eardrum, to vibrate. This vibration moves through the middle ear bones, whilst being amplified. This amplified vibration travels through the cochlea, stimulating the hair cells that are located along the spiral shaped cochlea. Different hair cells are stimulated depending on the frequency of the sound, where the auditory nerves in the cochlea are stimulated depending on the triggered hair cells. The auditory nerves, spiral ganglion axons, are connected to the cochlear nuclei in the brain stem. From the cochlear nuclei, the auditory nerve pathway continues to superior olive, inferior colliculus, specific thalamus and to auditory cortex in the brain.

Impaired hearing may be caused by any portion of the auditory pathway not functioning as it should. For example, the middle-ear portion may not function properly in which case hearing aid which amplifies sound are often used. In other cases the hair cells may be malfunctioning such that despite the vibrations being received from the middle ear, the hair cells are not triggered to activate any electrical responses to the vibrations. In such a case, electrical signals may be provided at the cochlear. Other portions of the auditory nerve path may be disconnected, and depending on which portion is malfunctioning, the electrical signals for neural stimulation may be provided at a location which would enable the signal to be received at the auditory cortex.

The electrical signals for neural stimulation may be provided by an implant interfacing with a portion of the auditory nerve path, starting from anywhere in the cochlea. FIG. 1 illustrates some of the main locations that the implant could interface the auditory nerve path, where each location is numbered. The labels in FIG. 1 relate to the steps of the auditory nerve path, where the path starts at “a”, which represents the cochlea, and eventually reaches “d”, which represents the auditory cortex. The potential locations for the implant interface is at the cochlear “a” with the spiral ganglion contained within the modiolus, at cochlear nerve “b” between the cochlear and the cochlear brain stem, at the brain stem “c” with the inferior colliculi for example, or at the auditory cortex “d”. Although an implant may be provided at one of the above mentioned locations in the auditory nerve path, in some cases implants may be provided at multiple locations to combine and maximise sensorineural function restoration. For example, an implant may be provided at the cochlea “a” as well as at the cochlear nerve “b”, or at the cochlea “a” as well as at the cochlear brainstem “c”. As will be understood by those skilled in the art, other combinations of implant locations may be used.

FIG. 2 schematically illustrates a first example of a system comprising an auditory implant 2 for providing stimulation of a portion of the auditory system. The target site selected for auditory stimulation may vary as discussed above. As shown in FIG. 2, the implant 2 includes a micro-electrode array 4 which includes a number of electrodes which protrude outside the housing of the implant so that they can be injected into the target site of the auditory system. A drive/read out integrated circuit 6 is provided for controlling the pattern of stimulation current driven through selected electrodes of the array 4. Optionally, the integrated circuit may also provide read out functionality for reading currents measured through the electrodes.

A wireless power receiver 8 is provided for receiving power supplied wirelessly by radio frequency communication from a wireless power supply unit 20 provided separately from the auditory implant 2. The wireless power supply unit 20 includes a power source 22, such as a battery or ambient energy harvesting unit (e.g. a solar cell), and a wireless power transmitter 24 for transmitting energy wirelessly to the receiver 8 in the implant 2. Any known technique for inductive or radio frequency energy transfer can be used. The wireless power supply unit could, for example, be worn on the user's head or body so as to be in proximity to the auditory implant 2.

The auditory implant 2 also includes a wireless data receiver/transmitter 10 for communicating data by wireless communication with a head mounted sound capture device 30. The wireless communication may be performed by any wireless technologies (e.g. Wifi®, Bluetooth®, etc.). The sound capture device 30 includes a sound capture unit 32 which includes microphone for capturing a sound, a processor 34 for processing the captured sound to generate a pattern of electrical stimulation to be applied to the micro-electrode array 4 within the auditory implant 2, and a wireless data transmitter/receiver 36 for transmitting sound information to the implant 2. The sound information indicates the pattern of stimulation to be applied based on the captured sound. The sound information received from the sound capture device 30 by the wireless data receiver 10 in the auditory implant controls the drive/readout integrated circuit 6 to apply the specified pattern of stimulation current to electrodes of the array 4. The stimulation currents may be applied simultaneously to a two-dimensional pattern of electrodes, rather than using a sequential raster scan. In alternative examples the processor 34 for deriving the pattern of electrical stimulation from the captured sound could be provided within the auditory implant between the wireless data receiver 10 and drive integrated circuit 6, instead of being in the head mounted sound capture device 30. Also in some examples the wireless power supply unit 20 and sound capture device 30 could be combined into one device.

FIG. 3 shows a second example of the implant 2, which in this example includes the sound capture unit 32 and processor 34 within the internal portion of the implant. For example, rather than comprising an external head mounted sound capture device 30 as shown in FIG. 2, the sound capture unit 32 of FIG. 3 may be provided as an internal implant. In one example, the vibration sensor 39 of the sound capture unit 32 may be located next to the middle ear portion if the middle ear is still functioning. More specifically, the vibration sensor 39 may be connected to the middle ear bones, where the vibrations are converted into electrical signals at the processor 34. The wireless power supply unit 20 is the same as in FIG. 2. With this example, the wireless data receiver/transmitter 10 is not essential as the sound can be captured within the internal implant. However it may still be desirable to provide the wireless data receiver/transmitter 10 for communicating with a separate data readout/control unit 40. For example, if the integrated circuit 6 is able to make current measurements through the electrode array 4, it may be desirable to transmit values derived from the readouts by the processor 34 to the remote data readout/control unit 40 so that these measurements can be analysed. Also, for control purposes of the implant, it may be possible to set certain configuration settings of the implant through a remote control unit 40 by wirelessly transmitting control signals to be received by the receiver 10 within the implant 2. The data readout/control unit 40 may have a processor 42 and a wireless data transmitter receiver 44 similar to the head mounted sound capture device 30 of FIG. 2 but may not have the sound capture unit 32.

FIG. 4 shows the implant of FIG. 2 or 3, where the arrangement of the implant is optimised for a cochlear implant. The individual micro-electrodes 104 are held within a block 105 or spacer material which extends along at least part of the length of each electrode. An example of a method for embedding the electrodes in the spacer material is described with respect to FIG. 26 below. In this case, the micro-electrodes 104 extend all the way through the block 105, such that the first end and second end of the micro-electrodes 104 extend beyond either sides of the block 105. For example, in FIG. 4, the second ends of the micro-electrodes 104 extend out of one end of the block 105 towards the drive/readout IC 6, and the first ends of the micro-electrodes 104 extend out of another end of the block 105 towards the flexible body 600 holding the micro-electrodes 104 within the cochlear implant. The flexible body 600 comprises openings at stimulation sites 601, where the first ends of the micro-electrodes 104 protrude out of the flexible body 600. This is illustrated in the enlarged portion of the upper part of FIG. 4. Although only four micro-electrode tips are illustrated as protruding out of the flexible body 600, it would be understood by the skilled reader that this is for schematic illustrations purposes only, and other number of micro-electrode tips may be protruding out of the flexible body 600 at each stimulation site. Similarly, only a small number of stimulation sites (where recording may also occur at these stimulation sites if sensing micro-electrodes are provided in addition to the stimulation micro-electrodes) are shown along the length of the flexible body 600, only for schematic illustrative purposes. The implant may comprise around 100 stimulation sites. In other examples, fewer or more stimulation sites may be provided depending on the requirements. Where some micro-electrodes are configured to be for sensing rather than stimulating, it is possible to detect changes in a number of different sites within the cochlea to assist implantation of the implant 2.

In some examples, the array of micro-electrodes 104 may also contain hollow-core channels. These hollow-core channels may be used to deliver fluids comprising cDNA gene construct which drive expression of brain-derived neurotrophic factor which in presence of electrical stimulation can promote the regeneration of spiral ganglion neurites, the hollow-core channels may be used as light delivery channels. For example, the lights delivered may be light in the visible or infrared spectrum for stimulating the nerves.

In some examples, the micro-electrodes 104 are not embedded in the block 105 and are directly contained within the flexible body 600 as a bundle of micro-electrodes. The flexible body 600 may be formed of flexible polymer.

In the lower part of FIG. 4, the implant in use is illustrated. As can be seen, the flexible tubular body 600 containing the micro-electrodes 104 are placed in the spiral shaped cochlea. The micro-electrodes 104 placed in the cochlea provide stimulation directly to the ganglion cell which bundle up and form auditory nerve of the cochlea. In general, different portions of the cochlea are sensitive to different frequencies. Accordingly, the stimulation site 601 comprising protruding tips of the micro-electrodes are located to interface with and excite the nerves at a particular location along the length of the cochlea.

As the micro-electrodes 104 are smaller in dimension than any electrodes currently used in known cochlear implants, it is possible to provide a greater number of channels for interfacing with the limited space available as target stimulation site. Furthermore, it is also possible to better control which area is to be stimulated. This is because the stimulation surfaces of the present invention are only several micrometers in diameter and thus the electrical field emanating from the stimulation surface is almost hemi-spherical and thus more predictable. That is to say, during stimulation, charges are distributed more evenly to the ganglion cells.

It is noted that although the embodiment shown in FIG. 4 illustrates the tubular body surrounding the micro-electrodes 4 as reducing in diameter, in other embodiments the diameter of the structure may be constant throughout the length of the tubular body. For example, around 100 micro-electrodes may be packaged into a tube having a diameter of approximately 300 μm. This is an average size of the diameter at the narrowest portion of known cochlear implants, which gives an appreciation as to how much smaller the micro-electrodes used in the present invention are in comparison to those currently used in the field, thus providing a thinner implant for the same count of channels as in prior cochlear implants using wider electrodes.

FIG. 5 illustrates another view of the micro-electrode array that may be used in a cochlear implant. An enlarged view of a first end tip of a micro-electrode that may be present at each stimulation site is also shown as a graph. At the first end the tips of the micro-electrodes comprises metallic cores that are electrochemically modified by the deposition of a layer of nano-structures. In this example, the nano-structures are made of gold, but they could also be formed of other metals or metal oxides. This deposition of the nano-gold layer is followed by a deposition of a redox reversible material for charge capacity improvement, such as iridium oxide. Also a secondary sensing modality specific to a detection analyte, such as glucose, may also be provided. The graph shows how varying the time for which the electrodeposition is performed affects the size of the gold nano-structure bumps formed on the end of the micro-electrode. The size of these gold nano-structure bumps relate to stimulation site dimensions, which implicitly charge storage capacity. As can be seen from the graph, the dimensions of the stimulation site at the tip of the micro-electrode can be linearly controlled as depicted in the graph, by modifying the electrodeposition length.

In the example demonstrated in FIG. 5, the overall diameter of the tubular body 600 comprising the bundles of micro-electrodes 104 is around 300 μm. The diameter of the tubular body 600 may be varied depending on the number of micro-electrodes 104 used, or the number of additional hollow-core channels required. The tubular body 600 in this example may have an overall length of 20 mm, comprising stimulation sites along its length at 400 μm pitch. However, the lengths between each stimulation sites may differ. For example, the length between two adjacent stimulation sites along the length of the tubular body 600 may become smaller as it moves away from the portion connected to the IC 6.

The stimulation sites may be arranged as a rectangular grid, as shown in FIG. 5. In other examples, the stimulation sites may be arranged in a circular or spiral shape.

In some examples, the implant may further comprise a removable stylet to aid the surgical procedure at insertion of the implant 2 into the cochlea through cochleotomy.

FIG. 6 is a scanning electron microscopy of a single micro-electrode in which the bend radius of the single micro-electrode is demonstrated. As can be seen, the individual micro-electrode has a bend radius of at least 500 μm. Such flexibility is useful in enabling the implant to fit easily and smoothly in the cochlea.

As in many conventional implant units, the main body of the implant 2 comprising the IC 6 may be configured to be placed in a recess formed in the temporal bone adjacent the ear of the individual receiving the implant.

FIG. 7 illustrates another example of a micro-electrode array used in the implant, particularly when the micro-electrodes are intended for interfacing with parts of the auditory nerve path that is not contained within the cochlea. The individual micro-electrodes 104 are held within a block 105 of spacer material which extends along at least part of the length of each electrode. The micro-electrodes 104 which are sharped for more efficient penetration at the first end are bonded to the integrated circuit 6 in the second ends, where the second ends are not shown in FIG. 7. As in the example of micro-electrode array of FIGS. 4 and 5, the micro-electrode array may further comprise hollow-core channels, and some of the micro-electrodes may be configured to sense. Accordingly, the implant may comprise an interface with multiple modalities and functions.

The stimulation or recording sites at the tip of the micro-electrode may all be in a single plane, or the tips may be protruding at different lengths. That is to say, the length of the micro-electrode protruding from the block 105 may differ.

FIG. 8 shows example arrangements of the micro-electrode array in the example illustrated in FIG. 7. For example, the protrusion shape may be applied to either of, or both of the individual micro-electrode tip profile and the overall profile of the tips of the micro-electrodes. That is to say, the tip of each micro-electrode may have a curved shape, or a triangular pointy shape, or a rectangular shape. Different shapes may be used depending on the function of each micro-electrode. For example, sharper pointed tip profiles may be useful for the micro-electrodes that are intended to do the initial penetration, or for a more accurate stimulation site. A round or flat profiled tip may be used for micro-electrodes that are for sensing. The overall profile refers to the general shape that the micro-electrodes form as a group. For example, if the tips of the micro-electrodes are all in-plane and lengths of the micro-electrodes that protrude out of the block 105 are the same, the overall profile may be considered to be rectangular.

It is noted that the overall profile may also be changed by changing the shape of the block 105 rather than the protruding lengths of the micro-electrodes. For example, if the block 105 is convex, the tips of the micro-electrodes located in the middle portion of the block may extend the furthest.

Different inter-shank arrangements may also be used for the micro-electrodes protruding from the block 105. The inter-shank arrangements of FIG. 8 can be considered a plan view looking into the axis that is parallel to the core of the micro-electrodes. Thus, the dots shown in the inter-shank arrangements can be considered as the tips of the micro-electrodes. As can be seen, the micro-electrodes may be provided in a rectangular grid-like shape, or a circular grid-like shape, or a cross shape. Different shapes may be adopted depending on the target stimulation site.

FIG. 9 shows various views of another example of a micro-electrode array used in the implant. In this example, the micro-electrodes have an overall profile which is triangular. More specifically, the protruding lengths of the micro-electrodes decrease gradually. A side view and a top view of the implant are shown. As can be seen from the top view, the inter-shank arrangement in this case is cross-like. This reflects the oval shape of the inferior colliculus that is the stimulation target site.

FIG. 10 shows an example of a portion of the array 4 of micro-electrodes (wire electrodes) of the auditory implant 2 in more detail. A number of electrodes 104 are arranged in a bundle with the electrodes 104 arranged alongside each other (for example the wire electrodes may be arranged parallel to each other or nearly parallel). The wires could be arranged in the bundle in a regular pattern (such as a square/rectangular lattice or stack arrangement, or a hexagonal packed arrangement), or in an irregular pattern. Each wire electrode 104 includes a core 106 made of a conducting material (e.g. a metal or alloy) surrounded by an insulating material 108. The electrodes 104 are ultramicroelectrodes (UMEs) having a diameter less than or equal to 30 μm. In this example the metal core 6 is made of gold, but other examples of conducting materials which could be used include copper, silver, gold, iron, platinum, lead or other metals, as well as crystalline or amorphous alloy compositions such as brass, bronze, platinum-iridium, lead-silver and magnetic alloys such as FeSiB. In this example, the insulating material 8 is glass, but other examples could use plastics or other insulators.

The electrodes 104 have a first end for interfacing with the target site for auditory stimulation. The target site could be a site within the cochlea or could be a site of the auditory system outside the cochlea, such as the cochlear nerve, the cochlear brain stem or the auditory cortex. The electrodes 104 also have a second end for interfacing with the integrated circuit which drives the stimulus currents and/or receives signals measured from the target site. At the first end, the wire electrodes 104 each have an impedance reducing layer of gold nano-structures deposited on the tips of the wire electrodes, and an iridium oxide (IrOx) functionalisation layer comprising a layer of iridium oxide nano-structures deposited on top of the gold nano-structures. At the second end, the tips of the wire electrodes have a connection layer for connecting to the integrated circuit. The connection layer in this example is also made of gold nano-structures, but the second end does not have the additional functionalization layer.

FIG. 11 shows an image of the second end before hemisphere deposition (upper image) and of the gold nano-structure hemispheres formed on the second ends of the wires (lower image). Each individual single crystal of the hemisphere may have a unit size in the nanometre scale, e.g. smaller than 300, 100 or 50 nm for example. On the other hand, the overall hemisphere of nano-structures on the second-end may have a width at the micrometre scale, e.g. around 10-20 μm in this example. As can be seen from FIG. 11, the hemisphere may extend over the insulating sheath of the wires as well as the core material on the second-end to facilitate contact with the integrated circuit providing the electronics for reading out signals from the electrodes. The hemispheres at the first end may be narrower and extend over a smaller percentage of the wire tip surface than the second end hemispheres. It will be appreciated that the gold-nanostructure layers formed at the first and second ends of the wire electrodes need not be perfectly hemispherical—in general any mound or bump formed on the tip of the wire electrodes may be sufficient.

FIG. 12 shows images illustrating the various layers deposited at the tips of the wire electrodes. The left hand image of FIG. 12 shows the bare polished metal core prior to depositing either of the layers onto the tip of the wire. The middle image shows the electrode after depositing the gold nano-structure layer. The layer of gold nanostructures has a flaky consistency, providing a large surface area for charge transfer which helps to reduce the impedance at the tips of the wire. The right hand image of FIG. 12 shows an image of the wire electrode after depositing the iridium oxide layer on top of the gold nano-structure layer.

The iridium oxide layer has a spongey consistency and provides a surface modification suitable for a range of biosensing or electrochemical applications. For example, the IrOx layer facilitates pH sensing. Also, the chemical properties of iridium oxide provide increased charge storage capacity which enables current injection for stimulation of the target site in the auditory nerve path.

For glass ensheathed ultramicroelectrodes (UMEs) to be used in any electrophysiological application which involves reception or transmission of electrical signal through any length, the following characteristics are advantageous:
a controllable frequency response input representing one side usually the one in contact with the biological/and or liquid sample,
a well-insulated and electrical conductive length/body and a low-ohmic connection on the other side, usually the connection side or second-end.
UME connection to any microscopic and/or macroscopic conductor by mechanical means either reversibly or non-reversibly preferably features a repeatedly deformable positively protruding mass from surface.

Glass ensheathed UMEs represent an ideal platform for auditory nerve stimulation because of their small size, massive scalability and ability to be interfaced with emerging high-channel count pixel drive/read-out technologies. UMEs feature small stray capacitances (e.g. less than 0.5 pFcm⁻²) given the high insulator-conductor ratio, mechanical workability, broad material choice and commercial availability. UMEs usually have one dimension in the micrometre or nanometre domain and at least one in the millimetre or centimetre region, thus the properties of the electrified interfaces are to be carefully considered when high frequency electrical signal need to be passed by micron-sized or nano-sized interfaces.

Current challenges in auditory stimulation and sensing miniaturisation are signal coupling and transport. The smaller the sensors are, the higher impedances (Z) become, resulting in significantly weakened signals and high noise levels. The interface's electrical coupling properties consequently bring limitations in the design of the drive/read-out electronics. Firstly, more amplifier stages and higher amplifier gains are required to condition the stimulus signals or recorded signals. Secondly, pre-filter and impedance matching circuitry are included to reduce ambient noise and pick up small-signals. Thirdly, power consumption of these additional amplifier stages could easily be a critical issue when limited power sources are available i.e. for battery powered implants—thus improving signal strength while keeping electrode dimensions in the micron and sub-micron domain is of paramount importance.

These issues can be addressed using a auditory implant with micro-electrodes as discussed above. By performing a two-step surface modification of the tips of the UMEs at the first end, to include both a highly fractalized flake-like gold nano-structure layer and a second layer of highly porous metal oxide (e.g. iridium oxide), the impedance at the first end of the electrodes can be greatly reduced. This is shown in the graph of FIG. 13 which compares the impedance across different frequencies for three probes:
“polished Au”—a probe made of bare gold metal wires without surface modification of the tips at the first end
“IrOx modified”—a probe where the tips of the wires at the first end have the IrOx layer but not the intervening gold nano-structure layer
“jULIE”—a probe with electrodes as in the example of FIG. 10 with both the gold nano-structure layer and the IrOx layer at the first end tips of the wires.
   As shown in FIG. 13, the impedance at the first end of the jULIE wires is an order of magnitude lower than the other types of wires. To test jULIEs we performed recordings in the olfactory bulb (OB) of anaesthetized mice (4-6 weeks old, Ketamine/Xylazine anaesthesia) using a Tucker Davis RZ2 amplifier with a PZ2 pre-amplifier and RA16AC-Z headstage. Extracellular spikes were reliably recorded with amplitudes of up to 1.6 mV. Consistent with this, when jULIEs were lowered several mm into the brain and returned to a superficial recording position, extracellular units were reliably recorded throughout the olfactory bulb. Due to the small size of the recording site and minimal damage to the tissue, jULIEs were found to be exceptionally suited for recording large amplitude (500-1500 μV), well isolated signals from the close vicinity of neurons (20-30 μm). FIGS. 14 and 15 show amplitudes of extracellular neuronal recordings made in a mouse brain using a typical commercially available probe and the jULIE probe respectively. As is clear from FIG. 15, the amplitudes recorded using the jULIE probe are much larger than the amplitudes shown in FIG. 14 for the commercial probe. Hence, signal to noise ratio can be improved and there is less need for additional amplification, helping to reduce power in battery-powered implants for example.

FIGS. 14-15 show results of using the electrodes for neuronal recording in the olfactory bulb. In the present invention, the micro-electrodes of the implant are arranged to closely match the anatomical structure. For example, as discussed above in relation to the embodiments illustrated in FIGS. 8 and 9, the lengths and the arrangement of the micro-electrodes may be determined based on the anatomical structure of the target stimulation site. Furthermore, in portions of the auditory nerve path such as the cochlear nerve, the auditory brain stem or the auditory cortex, the micro-electrodes may be inserted in a manner that the lengths of the micro-electrodes are at least in parts parallel with portions of the nerve cell axons of the target auditory nerve path site. Hence, these electrodes 4 can also be used for a auditory implant designed for auditory stimulation. By enabling a reduced impedance connection to the target site in the interface at the target stimulation (or reading) site, this reduces the need for as much amplification circuitry, hence saving power consumption and prolonging battery life (thus reducing the frequency that the user may need to recharge or even replace the battery).

Other advantages of the UME array 4 include:

(i) the dimensions of the penetrating wires are 2× to 5× times smaller and recording sites can be up to 50 times smaller (e.g. 1 μm) than in conventional probes. Also, the use of the Taylor-Ulitovsky method as discussed below results in wires with smoother sides than in conventional probes. This results in reduced tissue displacement and damage as well as in highly localized stimulation/recordings with better unit separation.
(ii) the nanostructured interface represents an excellent platform for further improved electrical coupling characteristics with the extracellular media, for example the nanosized gold/IrOx interface allows for substantially higher signal-to-noise with amplitudes of up to 1.5 mV compared to typically 200-500 μV with conventional electrodes.
(iii) the material choice enables semi-automatic preparation for stimulus sites pre-arrangement to fit anatomical structures; and needle-like sharpening for seamless penetration of tissue.
(iv) there are also substantially improved charge transfer capabilities i.e. enabling current injection for stimulation purposes, in a highly localized manner.

FIG. 16 is a flow diagram illustrating a method of manufacturing the array of micro-electrodes 4 for the implant. At step 120 wire electrodes are formed using the Taylor-Ulitovsky method. The Taylor-Ulitovsky method is a technique for forming glass-sheathed wire electrodes with a very fine diameter, e.g. as small as a few microns. In the process, the metal or alloy conducting material is placed inside a glass tube which is closed at one end and the other end of the tube is heated to soften the glass to a temperature at which the conductor melts. The glass can then be drawn down to produce a fine glass capillary with the metal core inside the glass. Hence, metal cores of diameters in the range 1 to 120 microns can be coated with a glass sheath a few microns thick with this method. In particular, wires with a core in the range 1-10 μm surrounded in 10-40 μm of glass can be useful for electrical and electrochemical sensing. The metal used can include copper, silver, gold, iron, platinum, lead or other metals as well as crystalline or amorphous alloy compositions such as brass, bronze, platinum-iridium, or magnetic alloys.

At step 122, the electrodes are formed into a bundle or stack with the wires running parallel to each other. For example, the microwires can be machine wrapped into bundles of 10s, 100s, 1000s, 10000s or 100000s of wire electrodes, to provide multiple channels for recording or cover the available contact portions on an integrated circuit.

At step 124, the relative positioning of the wire electrodes in the bundle may be adjusted using a magnetic field, as shown schematically in FIG. 17. As shown in the top part of FIG. 17, when the wire electrodes 104 with a substantially round cross-section are bundled together they will tend to pack together in a hexagonal packed arrangement, in which the electrodes in one row are offset relative to the electrodes in another row. However, for driving stimulus currents through the electrodes and/or reading out the signals measured using the electrodes, it can be useful to interface the wire electrodes with respective contact portions of an integrated circuit (IC), such as those used in multi-electrode arrays (MEA) and other pixelated integrated circuits. Most commercially available ICs have the pixelated contact portions of the readout circuits arranged in a two-dimensional square or rectangular grid pattern. Therefore, to match the industry standard rectangular contact point arrangement of an integrated circuit, it can be useful to reposition the wires in the bundle to form a square or rectangular grid pattern, in which the electrodes form rows and columns as shown in the right hand diagram at the top of FIG. 17. The lower part of FIG. 17 shows one technique by which this can be done. The wire electrodes 104 are wound through a passageway 130 having a square or rectangular cross section. For example, the passageway 130 may be a tube which could be enclosed on all four sides of the bundle, or could be missing one of the sides (e.g. winding the wires through the inside of a U-shaped bar can be enough). By applying a strong magnetic field (stronger than the electrostatics acting on the wires) to the wires as they pass through the gap, the relative positioning of the wire electrodes can be adjusted to form a square or rectangular grid array. For example, if the wires have gold cores, gold is a diamagnetic material and so a strong enough magnetic field can slightly repel the gold wires, and by pushing them up against the inside of a square or rectangular tube, this forces the wires into the desired square or rectangular grid arrangement.

Alternatively, step 124 can be omitted if the pitch of the wires within the bundle or the size of the gold contacts bumps at the second end of the wires will be sufficient that they can interface with a readout circuit regardless of the hexagonal packed arrangement.

At step 126 of FIG. 16, the bundle of wire electrodes is bonded together. This can be done in various ways. In one example, a filler material or adhesive may be introduced between the respective insulating sheets of the wire electrodes 104 to bond the electrodes together in the bundle. Alternatively, as shown in the example of FIG. 18, the wire electrodes can be bonded together by melting the insulating sheath of respective wires together so as to coalesce the insulator into a common matrix of insulating material surrounding the conducting wire electrodes. For example, this approach can be particularly useful when the insulating material is glass. Hence, as shown in the example of FIG. 18, the individual sheaths of the different wires are no longer visible and instead the wire electrodes are surrounded by a common insulating matrix of glass. This approach can be particularly useful for increasing channel density, because by avoiding the need to include a filler or adhesive between the respective wires, a greater number of electrodes per unit area can be included in the bundle.

Note that the wire electrodes do not need to be bonded along their full length. For example, it can be useful to leave a portion of the wire electrodes nearest the first end unbonded so that the free ends of the wire electrodes can spread out when inserted into a target sample, to increase the area over which recordings or current injection can be performed.

At step 128, a connection layer comprising metal nano-structures is deposited on the tips of the wire electrodes at the second end of the wires. The connection layer can be deposited by electrodeposition, in which the bundle of electrodes is held in a bath of electrolyte and a voltage difference is applied between the wire bundle and another electrode to cause ions in the electrolyte to be attracted to the wire electrode bundle, depositing a coating of metal nanostructures on the tip of each wire.

In one particular example, gold micro-hemispheres were deposited from a two-part aqueous cyanide bath containing 50 gL⁻¹ potassium dicyanoaureate(I) (K₂[Au(CN)₂]) and 500 gL⁻¹ KH₂PO₄ dissolved sequentially in deionized water (18 MOhm) (Tech, UK) at 60° C. All reagents were supplied by Sigma-Aldrich, UK, and were used without further purification. Prior to electrodeposition the polished substrate was washed with ethanol (90%), rinsed with deionized water, wiped with a lint-free cloth (Kimwipes, Kimtech, UK) and dried at 50° C. for 1 hour in an autoclave. The electrodeposition protocol was carried out with a VSP 300 potentiostat-galvanostat (Bio-Logic, France) controlled with EC-Lab (Bio-Logic, France) in a three-electrode cell setup composed of a gold UME bundle as working electrode (W_{E}), a coiled platinum wire (99.99%, GoodFellow, US) as counter electrode (C_{E}) and a Ag/AgCl|KCl/_{3.5M} reference electrode (REF) supplied by BASi, USA (E vs. NHE=0.205V). The REF was kept separated from the bath by a glass tube containing the support electrolyte and a porous Vycor glass separator. During gold deposition the W_{E} potential was kept at E_{red}=−1.1V vs. REF for a time determined according to the desired size of the gold hemisphere to be formed. During electrodeposition the bath was thermostated at 60° C. under vigorous (500 rpm) stirring. This technique has been successful for many different types of metal conductor material, including gold, platinum, tin, copper, brass, bronze, silver and lead.

FIG. 19 is a graph showing how varying the time for which the electrodeposition is performed affects the size of the gold nano-structure bumps formed on the end of the wires. The upper line in FIG. 19 shows variation of the width of the gold bumps with electrodeposition time, and the lower line shows variation of the height of the bumps with electrodeposition time. Hence, the size of the gold bumps can be carefully controlled by varying the electro-deposition time.

Gold can be a particularly useful material for the second end connection layer. In contrast with their applications for the first end sensing, the connection of individual or high-count bundled UMEs to integrated circuitry is poorly examined and represents a significant drawback towards their usability in biomedical applications. In known interfacing methods for bonding individual or UMEs to macroscopic conductors or integrated circuitry, the main practices are based on soldering, conductive silver-epoxy bonding or mercury-dip. Although applied, these methods can easily increase the RC cell time constant at high frequencies given the stray capacitance at the glass-mercury/conductive epoxy junctions and are not relevant for reversible contacting individual or bundled UME assemblies; scaling such practices to high-count UME bundles (up to 1 million, for example) are a considerable engineering challenge. The state-of-the-art indium bump bonding developed for pixelated sensor and read-out chip interconnection employing photolithography, sputtering and evaporation or later electrodeposition could be a suitable processing practice, however due to indium's tensile and ductile properties, mechanical properties and overall tribological behaviour it cannot be applied as a reversible interconnection material in UME interfacing. Copper bumps as interconnects could be considered from a mechanical point of view, however given their possible diffusion into SiO in the presence of an electric field, breaking down transistor reliability, and affinity towards oxidation, make Cu a less attractive candidate as an interconnect material in physiological environments.

In contrast, gold is a promising contact material in medium wear conditions which can seamlessly enable reversible, scalable, low-cost, ultra-fine pitch and high yield bumping for interconnection purposes.

At step 132 of FIG. 16, an impedance reducing layer of metal or metal oxide nano-structures is deposited on the tips of the wire electrodes at the first end. This can be done by the same electrodeposition protocol as described above for step 128 for the second end. The material used for the nano-structures at the first end can be the same or different to the material used for the nano-structures at the second end, but in one example both use gold nano-structures.

At step 134 a functionalization layer is deposited on the impedance reducing layer at the first end. Again, this can be deposited by electrodeposition (although other techniques such as spraying could also be used). For example, a layer of metal oxide (e.g. iridium oxide) can be deposited on top of the gold nano-structures at the first end.

In one particular example, the electrodeposition protocol was carried out from a modified electrolyte solution based on a formulation reported by Meyer et al. (2001, “Electrodeposited iridium oxide for neural stimulation and recording electrodes”, Neural Systems and Rehabilitation Engineering, IEEE Transactions on, 9(1), pp.2-11.), containing 10 gL⁻¹ iridium (IV) chloride hydrate (99.9%, trace metal basis, Sigma-Aldrich, Germany), 25.3 gL⁻¹ oxalic acid dihydrate (reagent grade, Sigma-Aldrich, Germany), and 13.32 gL⁻¹ potassium carbonate (99.0%, BioXtra, Sigma-Aldrich, Germany). Reagents were added sequentially to 50% of the solvent's volume first by dissolving IrCI in the presence of oxalic acid followed by the addition of K₂CO₃ over a 16 hour period until a pH=12 was reached. The electrolyte was aged for approximately 20 days at room temperature in normal light conditions until the solution reached a dark blue colour. IrOx was electrodeposited using a multichannel VSP 300 (Bio-Logic, France) potentiostat-galvanostat in 3 electrode cell setup comprising a glass-ensheathed Au wire bundle as working electrode (WE), a platinum rod (0.5 mm diameter, 99.95%, Goodfellow, US) as counter electrode, and Ag|AgCl|KCl/_{3.5M} (Bioanalytical Systems, US) as a reference electrode (REF). The electrochemical protocol was composed of three consecutive stages combining galvanostatic polarisation (GP), cyclic voltammetry (CV) and pulsed potentiostatic protocols (PP). Between protocols open circuit voltage (OCV) of the WE was monitored for 180 second and represents the steady-state period. During galvanostatic deposition the WE potential was set to 0.8V vs. REF for 500 seconds. During CV deposition the WE potential was swept from −0.5V to 0.60 V vs. REF at 100 mVs in both anodic and cathodic direction. During the pulsed potentiostatic deposition the WE potential was stepped from 0V to 0.60V vs. REF with 1 seconds steps for 500 seconds.

As shown in FIG. 20, the tips of the wire electrodes at the first end can be polished/sharpened to provide a tapered surface that is angled to a point, to facilitate insertion into the target site within the auditory nerve path.

The method of FIG. 16 may include an additional recess forming step 136 between steps 120 and 122. In step 136, part of the tips of the electrode is dissolved using a solvent to form a recess 140 in the end surface of the electrode 104 as shown in part a) of FIG. 21. For example, the recess can be formed by an electrochemical leaching step (e.g. by dissolving into an electrolyte in the presence of electrical current). The parts of the electrode 104 which are not to be dissolved may be masked by covering them with a mask material, so that only the portion at the end of the tip is dissolved. The subsequent steps of FIG. 16 are then performed on the wire electrodes having the recess in their tips. Therefore, as shown in part b) of FIG. 21, when the impedance reducing layer is subsequently deposited at step 128 of FIG. 16, the nano-structures 142 are deposited on the inside of the recess 140. The nano-structures 142 may also extend onto the surface of the electrode tip outside the recess. When the functionalization layer (e.g. IrOx) is then deposited on top of the impedance reducing layer at step 134, the functionalization layer 144 is deposited inside the recess. The functionalization material may also protrude out of the recess beyond the end of the electrode tip as shown in part c) of FIG. 21.

The approach shown in FIG. 21 provides several advantages. Firstly, providing a recess means that a greater volume of iridium oxide or other functionalization material can be deposited at the end of the electrode, which can improve the electrochemical properties of the electrodes. For example, given the available space, the charge capacity of the iridium oxide layer can be improved up to a 1000 times. Also, this approach provides robustness against mechanical deterioration of the electrode tips. During their working life, the electrodes may repeatedly be inserted into a sample and removed, and so the tips of the electrodes may gradually be worn away by contact against the sample, which can cause deterioration of the signals measured by the electrodes. By including the recess and depositing the surface layers inside the recess, then even if the end of the electrodes is worn down (e.g. so that the surface now is at the position indicated by the line 146 in FIG. 21), then there will still be a layer of the impedance reducing nano-structures and a layer of the functionalization material at the end of the electrodes, so that the electrode can still perform its function. Therefore, the recessed design helps to increase the electrodes' lifetime.

A similar recess may be formed at the second end of the electrodes, with the connection layer of metal nanostructures formed at least partly inside the recess. Again, this helps provide robustness against mechanical deterioration, which could otherwise wear away the connection layer if the connection layer is repeatedly pressed against the contact bumps of pixel readout circuits as discussed below.

The integrated circuit 150 used to drive stimulation currents and/or read out and amplify signals measured using the UME array 4 can be implemented in different ways. For stimulation purposes, the IC may be a pixelated display driver, such as the CMOS based circuits for driving LED or OLED displays. For readout of measured signals, the IC 50 may be a multi electrode array (MEA), a CMOS based potentiostat or a pixelated photodetector. All these are already available commercially and therefore it is not necessary to design a bespoke circuit for this purpose, which reduces the cost of implementing an apparatus for electrochemical measurements. If both stimulation and readout is required, then a subset of pixels of the IC corresponding to the stimulation electrodes may be implemented similar to a pixel of a display driver, while another subset of pixels of the IC corresponding to readout electrodes may be implemented similar to an element of a multi-electrode array, CMOS based potentiostat or pixelated photodetector. FIG. 22 shows how the micro-electrode array 4 may be interfaced with the integrated circuit 150. As shown in FIG. 22, the gold contact bumps 158 at the second ends of the respective wire electrodes 104 can simply be pressed directly against the contact bumps 154 of the respective pixel driving/readout circuits 155 of the IC 50 to provide the electrical connection between the wires and corresponding pixels (without any interposing connector unit between the wire bundle and the IC 150). Hence, the integrated circuit provides a multi-channel amplification and readout system for reading the electrode signals from the respective wires. It is not essential that every pixel driving/readout circuit of the IC 150 interfaces with a corresponding wire electrode. Depending on the arrangement of the wires within the bundle, it is possible that some pixel readout circuits may not contact a corresponding wire.

An alternative is that the wires can also be embedded in a second cladding holding wires together and have the bumps for connection.

For wire bundles with relatively low channel count (e.g. less than 10000 wires in the bundle), it may also be possible to individually bond each wire to connectors in the integrated circuit. However, when the channel count is higher (e.g. greater than 10000 wires), then it becomes increasingly impractical to individually bond each wire, and in this case the approach shown in FIG. 22 may be more useful, whereby the bumps on the end of each wires are simply pressed against the contact portions of a pixelated integrated circuit.

In the above examples, the functionalization layer is made of iridium oxide. However, this is just one example and other types of functionalization layer could also be used. The functionalization layer may be selected according to the intended purpose of the respective electrodes. Different electrodes of the array could have different types of functionalization layer deposited on top of the impedance-reducing gold nano-structure layer at the first end of the electrodes. For example, the same array could include a mixture of electrodes designed for auditory stimulation (e.g. with IrOx functionalisation layer) and electrodes designed for sensing target substances or conditions. One advantage of using gold as the nano-structure impedance reducing layer is that gold nano-structures provide a good platform for a range of different functionalization layers for different biosensing or electrophysiological purposes. For example, the functionalization layer may include other metal oxides, such as copper oxide (useful for sensing glucose) titanium dioxide or manganese oxides, or could include other substances such as carbon nanotubes, graphene, ATP, DNA or other nucleic acids, proteins etc.

Another example of a functionalization layer may comprise self-assembled monolayers. Self-assembly describes the spontaneous formation of discrete nanometric structures from simpler subunits. During the process of self-assembly, atoms, molecules or biological structures form a more complex secondary layer with fewer degrees of freedom due to packing and stacking. The simplest self-assembled systems are self-assembled monolayers (SAMs). SAMs are formed by the adsorption of molecules on solid surfaces and are governed by intermolecular forces. The most popular molecules forming SAMs are thiols and dithiols: in biology and medicine these molecules are used as building blocks for the design of biomolecule carriers, for bio-recognition assays, as coatings for implants, and as surface agents for changing cell and bacterial adhesion to surfaces. Hence, by covering the layer of metal or metal oxide nanostructures (e.g. the nano-rough gold deposits) on the tip of the microwires with SAMs, we can functionalise the tip and build up a highly specific bio-sensitive layer. This can enable the identification of DNA fragments, biomolecules or analytes present in tissue, bodily fluids, or nerves.

Hence, by providing different functionalization layers, the implant may function as a wide variety of electroceutical devices (devices which employ electrical stimulation to affect or modify functions of the body) or instruments for recording data about electrical or electrochemical properties of the sample in which the electrodes are inserted. In some examples, the auditory implant may have a dual function, acting as both an electroceutical device and a recording instrument. Alternatively, other embodiments could solely act as a auditory implant with no recording functionality.

Where different types of micro-electrodes are bundled together (e.g. some stimulation electrodes and other electrochemical sensing electrodes), different approaches may be used to assemble the different types of electrodes in the implant. In one example, the array 4 may be formed from a number of different subsets of electrodes, each subset having different types of functionalization layer deposited on the top of the gold nano-structure impedance reducing layer at the first end of the wires. For example, a number of bundles may be manufactured separately using the process described above, each having a different type of functionalization layer, and then the respective bundles can be assembled into a probe, to allow a single probe to make two or more different types of electrochemical measurements. For example, a bundle may be provided with iridium oxide functionalization layer may provide stimulating currents for auditory stimulation or may sense pH or electrical currents, while another bundle may have copper oxide or graphene functionalised wires for glucose sensing, etc. The bonding of the different subsets of wire electrodes could be done through an adhesive or a filler layer, or by melting the glass insulating sheaths of the wires together.

In another alternative, instead of forming the respective bundles 82 separately and then assembling them together, a single wire bundle could be made with different functionalization layers on respective wires of the bundle, for example by masking some wires during the step of depositing the functionalization layer or by ensuring that the electrodeposition current is only applied to some of the wires, with multiple functionalization deposition steps for the different types of functionalization layer.

As shown in FIG. 23, in addition to the wire electrodes 104 having a conducting core 106 surrounded by an insulating sheath 108, the micro-electrode array 4 may also include hollow-core channels 170 which comprise a hollow core (made of air) surrounded by a sheath of the same insulating material 108 as used for the wire electrodes 104. The hollow-core channels 170 are arranged parallel to the wire electrodes 104. For example, the hollow-core channels 170 can be formed by providing some wires in the bundle with a solid core of soluble material clad in an insulating sheath, and bundling these together with the conducting-core electrodes 104 in a desired pattern, and then dissolving the core of the hollow-core channels 170 to leave an empty void at the centre of these channels. The hollow-core fibres 170 can be useful for both delivery and extraction of liquid or almost liquid phase substances from the surface or interior of virtually any biological media. For example, they could be used as micro-fluidic channels for local delivery of pharmaceuticals, molecules, cells, genes, tissue. For example, the implant 2 could include a reservoir from which the substance to be supplied into the tissue is drawn by the hollow-core channels 170 of the array 4. The substance to be delivered can be injected into the reservoir by a needle when the implant is in position.

FIG. 24 shows an example in which the wire electrodes 104 (including both their core 106 and the insulating sheathes 108) are partially embedded in a block of cladding material 250 along part of their length, with gaps 252 between the respective wire electrodes along a remaining part of their length. The layer of nanostructures or other modifications at the tips of the electrodes are not shown in FIG. 24 for conciseness, but it will be appreciated that the tips of the electrodes can be modified in the same way as in any of the examples described above. The cladding material 250 provides a rigid support for the electrodes to prevent separation of the bundle of wire electrodes 104, while providing gaps between the free ends of the electrodes 104 can be useful for allowing some separation of the wire electrodes when inserted into a sample (e.g. the insulating material may be a flexible material), increasing the area over which the wire electrodes can gather measurements or provide stimulation. Although FIG. 24 shows an example where the cladding material 250 is formed at the second end of the probe, in other examples the cladding material 250 could be at a mid-point of the electrodes so that both ends of the wire electrodes may be free to move as there are gaps between the insulating sheathes of the wires.

FIG. 24 shows an example process for manufacturing the probe with flexible wire electrodes 104 held together by a block of rigid cladding material 250. As shown in the left hand part of FIG. 24, initially the wire electrodes may be formed individually with the core 106 surrounded by a first sheath 108 of insulating material and a concentric second sheath 256 of cladding material outside the first sheath 108. The cladding material may be more soluble to a given solvent than the insulating material used for the first sheath 108. Other than the solubility, it is preferable if the cladding material is similar in physical properties to the insulating material, e.g. similar in thermal expansion and composition. The wires 104 are bundled together parallel to each other, and heat is applied to melt the second cladding layer 256 together to form a block of melted-together cladding 250 with the wires 104 of core material 106 and insulating material 108 embedded inside the cladding block. A solvent which can dissolve the cladding but not the insulation is then applied to dissolve part of the cladding block 250 down to a given level, preserving the initial ordering and parallel stacking of the wires and leaving the free end of the wires with gaps 252 in between while a bound section of wires is surrounded by the cladding block 250. These steps are valid in a range of possible geometries, e.g. hemispherical, saw-like, planar, random or combined.

In summary, by providing a auditory implant comprising a number of micro-electrodes made of conducting material and insulating material surrounding the conducting core, with a layer of metal or metal oxide nano-structures deposited on the tips of the wire electrodes at the first end of the wire bundle, this can provide much lower impedance, increasing the signal/noise ratio of currents transmitted for stimulation purposes. The core material may be one of gold, platinum, copper, brass, nickel, tin, silver, iron, lead, brass, bronze, platinum-iridium, silver-lead for example. The insulating material may be glass or plastic, for example.

In some examples the wire electrodes may have separate insulating sheaths of the insulating material. Alternatively, the wire electrodes may be disposed in a common insulating matrix of insulating material, which can be formed for example by melting the glass sheaths of the individual wire electrodes together as discussed above.

The metal or metal oxide used for the nano-structures in the impedance reducing layer could be any of the following: gold, platinum, ruthenium, titanium, iridium, indium, manganese, or oxides of such materials such as manganese oxide or ruthenium oxide. In particular, using nano-structures made of a noble metal, such as gold or platinum, can be particularly useful for reducing the impedance. In particular, gold nano-structures plus a thin layer of IrOx have been found to be particularly effective as shown in the graph of FIG. 13.

The layer of metal or metal-oxide nano-structures formed at a first end of the wire bundle may act as an impedance reducing layer for reducing impedance at the interface with the sample being measured, and also can act as a surface for functional modifications.

Also, at the second end of the micro-electrodes there may be a connection layer of metal nano-structures on tips of the wire electrodes. This end is used as the second end of the probe for outputting signals to readout electronics. The nanostructures in the connection layer at the second end of the wire bundle may be made of the same material as the nanostructures in the impedance reducing layer at the first end, or alternatively the respective ends of the wire bundles may be provided with nanostructures of different materials (e.g. gold at the first end of the probe and platinum at the second end). Metal oxides may be less preferred for the second end as they may increase the resistance of the electrical connection, whereas for the first end they may be useful at bringing down impedance because of the increase in the specific surface given their fractalised porous structure. For the second end, a nanostructured surface can be advantageous because on compression it compacts instead of cracking.

In some examples, a layer of the metal or metal oxide nano-structures may be formed at the first end of the wire bundle, and a layer of metal nano-structures may be formed at the second end of the wire bundle.

One or both of the layers of nano-structures formed at the first and second ends of the wires may be formed from separate bumps of nano-structures, with each bump formed on the tip of a respective wire electrode. The bumps may have a rounded or hemispherical profile.

A functionalization layer may be deposited on the layer of metal or metal oxide nano-structures at the first end of the wire bundle.

For at least one of the wire electrodes, the tip of the electrode at the first end may comprise a recess, the layer of nano-particles may be deposited on the inside of the recess. If provided, the functionalization layer may be formed on top of the layer of nano-particles inside the recess. The functionalization layer may protrude out of the recess.

The wire bundle may in some examples comprise a first subset of wire electrodes with a first type of functionalization layer deposited on the layer of nano-particles at said first end of the bundle, and a second subset of wire electrodes with a second type of functionalization layer deposited on the layer of nano-particles at said first end of the bundle. In some cases there may be three or more subsets of wire electrodes with different types of functionalization layer.

In one example, the wire electrodes may be ultramicroelectrodes (UMEs). The wire electrodes may have a diameter less than or equal to 30 μm. In other examples, the wire electrodes may be even narrower, for example with a diameter less than or equal to 25 μm, less than or equal to 20 μm, less than or equal to 15 μμm, less than or equal to 10 μm or less than or equal to 5 μm. By providing an implant with smaller diameter wires than previous techniques, this enables a great increase in channel count in an implant of a given size, and also means less current needs to be transmitted through a given wire (the reduced impedance at the first end also helps reduce the current required), so that less tissue damage may be caused.

The nanostructures in the respective layers at each end of the probe may have a unit width less than or equal to 500 nm. More particularly, the unit width may be less than or equal to 400 nm, less than or equal to 300 nm, less than or equal to 200 nm, less than or equal to 100 nm or less than or equal to 50 nm. The term “unit width” refers to the width across the longest dimension of an individual nano-structure (e.g. an individual flake, grain or nanoparticle), not the width of the mass of nano-structures as a whole. In some cases the nano-structures at the first end may have a unit width which is less than or equal to 20% of the wire diameter of the electrodes, less than or equal to 15% percent of the wire diameter, less than or equal to 10% of the wire diameter, or less than or equal to 5% of the wire diameter. The nano-structures at the second end can also be less wide than the wire diameters of the electrodes, or alternatively can cover a greater area of the tips of the wire electrodes, and could cover the entire tip. Note that the different nano-structures within the layer will in practice have different unit widths to each other, but all the nano-structures may have a unit width defined within the thresholds described above. Similarly, the functionalization layer may also comprise a layer of nano-structures (e.g. of Iridium oxide, or another material), which may have unit widths as defined within the thresholds described above. The nano-structures in the functionalization layer may be of a different size to the nano-structures in the impedance reducing layer or connection layer.

The tips of the wires may be sharpened, or have a tapered end which meets at a point, to facilitate insertion into the target site in the auditory nerve path.

Another aspect of the invention relates to an implant as described herein for performing a diagnostic or therapeutic operation on a subject. Another aspect of the invention relates to a method of using an implant as described herein for performing a diagnostic or therapeutic operation on a subject. Preferably, the subject is a human or animal subject, more preferably, a human. Preferably, for this embodiment, the method comprises introducing the implant into the subject, for example in the cochlea, or with the electrodes inserted into other parts of the auditory nerve path, such as directly to the cochlear nerve, auditory brainstem or the auditory cortex. More preferably, the method further comprises the steps of transmitting current into the subject using the implant and/or reading out current from the subject using the implant.

In another embodiment, the invention relates to a method of delivering a drug to a subject, said method comprising contacting the subject with an implant as described herein, wherein the drug is present in or on the implant. Preferably, the drug is released at a localised target site in the subject. By way of illustration, drug molecules can be delivered to a target site via hollow channels in parallel with the stimulation electrodes or readout electrodes. In one embodiment, the drugs are delivered in liquid form, e.g. by injection into a reservoir within the implant, and are released through the hollow channels into the target site. Alternatively, drug molecules can be attached to the surface of one or more micro-electrodes via a linker moiety or other substance which can be activated by supplying a current pulse to the probe to release the drug. Another aspect of the invention relates to an implant as described herein which is used in conjunction with one or more therapeutic agents. Thus, another embodiment relates to an implant as described herein for use in treating a proliferative disorder in a subject, wherein said implant delivers one or more therapeutic agents to the subject.

In another aspect of the invention, the implant described herein is for, or for use in, the detection of various substances in a sample, or in a subject, including but not limited to, protons (e.g. detecting pH), biological substances (e.g. proteins, glucose, neurotransmitters, hydrogen peroxide, tissue modulators, calcium, nitric oxide, DNA/RNA, immunological molecules) and/or toxins (e.g. heavy metals). Preferably, the detection takes place in vivo. Thus, in one embodiment, the invention relates to a method of detecting a substance as defined above in a subject, said method comprising contacting the implant with a tissue or organ of the subject. By way of illustration, in order to detect or sense a change in pH, the electrodes can be modified using iridium oxide, for example, by coating at least a portion of the electrodes with iridium oxide. For alcohol detection, the electrodes can be functionally modified with one or more alcohol oxidases, for example, by attaching an alcohol oxidase to the probe using a suitable linker moiety. Other substances/molecules (e.g. enzymes, proteins, RNA, DNA, oligonucleotides, graphene, copper oxide, etc.) can also be attached to the surface of the micro-electrode tips in a similar manner. The skilled person would understand that selected electrodes of the implant can be tailored to detect a particular substance or molecule and would be familiar with literature methods for functionally modifying the surface of the electrodes.

In another aspect, the invention relates to one or more diagnostic methods using the implant described herein. For example, in one embodiment, the implant can be used to detect changes in the subject, such as changes associated with diabetes, including by detecting changes in the level of one or more biomarkers (e.g. glucose), for example in the bloodstream of the subject.

As discussed above, one benefit of the cochlear implant discussed above is that the use of micro-electrodes of the type discussed above means that it is possible to pack many wires into a small space. FIG. 25 shows a scanning electron microscope (SEM) image of a side view of the micro-wires of a cochlear implant, in which the bundle comprises approximately 100 micro-electrodes packed into a region of diameter approximately 300 μm. FIG. 26 is a SEM image showing a front view of the ˜100-channel micro-wire bundle. In contrast, presently available cochlear implants typically are 2-4 mm thick and only able to provide approximately 20-38 channels and usually the tip is of similar diameter (˜300 μm) where there is one or zero stimulation sites. Hence, the cochlear implant based on micro-wires as discussed above can provide a significant increase in resolution. It can also enable less drastic surgical intervention, because the micro-wires extend in parallel for a relatively long length (e.g. 5-10 cm) and are interfaced with an integrated circuit of similar dimension to the bundle of wires, so the cochlear implant need not increase in width across the length of the implant. This can help to reduce the likelihood of trauma during surgery to insert the implant causing loss of any residual hearing remaining in the patient.

Another advantage of the cochlear implant is that, due to the high channel count and small dimension, it becomes feasible to make more focused stimulation of certain regions of the cochlea. For example, it has been suggested that, for providing stimulation to boost understanding of speech or music, it would be useful to be able to make a targeted stimulation of a region at the top of the cochlea, which is responsive to relatively low frequencies. FIG. 27 is a micro-CT image of the implant with ˜100 channels as shown in FIGS. 25 and 26 inserted into the base of the cochlea of a gerbil. As shown in FIG. 27, the width of the micro-wire bundle is small in relation to the overall size of the cochlea, and so this shows it is feasible for the implant to make a focused stimulation of a specific site within the cochlea with signals at a specific frequency band. FIG. 27 also shows there would be space for two or more micro-wire bundles to be inserted into different sites of the cochlea, for specific stimulation of each site. Alternatively, a single micro-wire bundle could be formed of a number of sub-bundles which are designed to split from other sub-bundles and spread out as the implant is inserted into the cochlea, so that different patterns of stimulation can be applied at different sites in the cochlea.

FIG. 28 shows a micro-CT image of an implant with a micro-wire bundle having approximately 1000 channels, inserted into a sheep tympanic bulla. A sheep cochlea is of similar dimensions to a human cochlea, so this demonstrates that there is space for a 10- or 100-times increase in resolution in sheep or human cochlea in comparison to the gerbil cochlea. As shown in FIG. 28, there is space for further expansion of the number of channels if desired.

Hence, FIGS. 27 and 28 show that it is feasible to provide an auditory implant which support much greater resolution in stimulation than is possible with current implants. While FIGS. 27 and 28 show examples of inserting the micro-electrodes into a cochlea, in other examples the implant could be used to directly stimulate the auditory nerve, bypassing the cochlea.

Although illustrative embodiments of the invention have been described in detail herein with reference to the accompanying drawings, it is to be understood that the invention is not limited to those precise embodiments, and that various changes and modifications can be effected therein by one skilled in the art without departing from the scope and spirit of the invention as defined by the appended claims.

1. An auditory implant comprising:
an array of micro-electrodes including at least two stimulation micro-electrodes each comprising:
a core of conducting material,
insulating material surrounding the core, and
a layer of metal or metal oxide nano-structures deposited on tips of the micro-electrodes at a first end for interfacing with a target site for auditory stimulation; and
an integrated circuit to control a pattern of stimulation current driven through the at least two stimulation micro-electrodes.

2. An auditory implant according to claim 1, in which the micro-electrodes have a diameter less than or equal to 30 μm.

3. (canceled)

4. An auditory implant according to claim 1, in which the nano-structures at the first end comprise nano-structures made of a noble metal.

5. An auditory implant according to claim 4, in which the noble metal comprises gold.

6. An auditory implant according to claim 1, in which the at least two stimulation micro-electrodes also comprise a functionalisation layer deposited on the layer of metal or metal oxide nano-structures at the first end.

7. An auditory implant according to claim 6, in which the functionalisation layer comprises iridium oxide.

8. An auditory implant according to claim 1, in which the insulating material comprises glass.

9. An auditory implant according to claim 1, in which the micro-electrodes are mechanically flexible.

10. An auditory implant according to claim 1, in which the at least two stimulation micro-electrodes comprise Taylor-Ulitovsky wires.

11. An auditory implant according to claim 1, in which at least two protruding tips of the micro-electrodes at the first end have different lengths.

12. An auditory implant according to claim 1, in which tips of the micro-electrodes at the first end have at least one of the following shapes: a flat tip surface, a flat tip surface which is at least partially angled to the length of the micro-electrode, a curved tip surface, a polished angled tip surface with a sharpened point;
wherein for the at least two stimulation micro-electrodes the metal or metal oxide nano-structures are formed on the tip surface.

13. An auditory implant according to claim 1, in which the integrated circuit is configured to perform simultaneous driving of current through stimulation micro-electrodes arranged in a two-dimensional pattern.

14. An auditory implant according to claim 1, in which the integrated circuit is configured to receive sound information indicative of a captured sound, and to control the pattern of stimulation current based on the received sound information.

15. An auditory implant according to claim 14, comprising a wireless communication unit to receive the sound information from an external sound capture device.

16. An auditory implant according to claim 14, comprising a sound capture unit to capture sound and processing circuitry to process the captured sound to generate the sound information based on the captured sound.

17. An auditory implant according to claim 14, comprising processing circuitry to detect features in the captured sound indicated by the sound information and to determine a pattern of stimulation current to be applied to the at least two stimulation micro-electrodes based on the detected features.

18. (canceled)

19. An auditory implant according to claim 17, in which the processing circuitry is configured to modulate at least one of a spatial position, frequency, and intensity of the stimulation current applied to the at least two stimulation micro-electrodes based on the detected features.

20. An auditory implant according to claim 1, in which the array also comprises at least one sensing micro-electrode, and the integrated circuit is configured to control supply of current to, or readout of voltage or current from, the at least one sensing micro-electrode for sensing a predetermined substance or condition.

21. An auditory implant according to claim 20, in which the at least one sensing micro-electrode comprises any one or more of the following:
at least one glucose or pH sensing micro-electrode for detection of glucose or pH:
at least one sensing micro-electrode having, deposited on the layer of metal or metal oxide nano-structures at the first end, a functionalisation layer for supporting detection of said predetermined substance or condition;
at least one micro-electrode of the array which functions as both a stimulation micro-electrode and a sensing micro-electrode.

22-26. (canceled)

27. An auditory implant according to claim 1 comprising a mechanically flexible tubular body carrying the micro-electrodes, wherein the plurality of the micro-electrodes protrude out of the tubular body at individual stimulation sites located along the tubular body.

28-42. (canceled)
